SEQUENCE LISTING (50 sequences)

SEQ ID NO: 1 - RNA, 21 nt, synthetic construct
  source 1..21: mol_type = other RNA
gcagaaatgattgcactattt

SEQ ID NO: 2 - RNA, 21 nt, synthetic construct
  source 1..21: mol_type = other RNA
atagtgcaatcatttctgctt

SEQ ID NO: 3 - RNA, 21 nt, synthetic construct
  source 1..21: mol_type = other RNA
ctgctactcttcagcattatt

SEQ ID NO: 4 - RNA, 21 nt, synthetic construct
  source 1..21: mol_type = other RNA
taatgctgaagagtagcagtt

SEQ ID NO: 5 - RNA, 21 nt, synthetic construct
  source 1..21: mol_type = other RNA
cagtcccacttgtgtcaaatt

SEQ ID NO: 6 - RNA, 21 nt, synthetic construct
  source 1..21: mol_type = other RNA
tttgacacaagtgggactgtt

SEQ ID NO: 7 - RNA, 16 nt, synthetic construct
  source 1..16: mol_type = other RNA
cttttgacctgctaat

SEQ ID NO: 8 - RNA, 21 nt, synthetic construct
  source 1..21: mol_type = other RNA
attagcaggtcaaaagtgaac

SEQ ID NO: 9 - RNA, 16 nt, synthetic construct
  source 1..16: mol_type = other RNA
tcaagcccatctattt

SEQ ID NO: 10 - RNA, 21 nt, synthetic construct
  source 1..21: mol_type = other RNA
aaatagatgggcttgactttc

SEQ ID NO: 11 - RNA, 16 nt, synthetic construct
  source 1..16: mol_type = other RNA
ctccgctgaccttaaa

SEQ ID NO: 12 - RNA, 21 nt, synthetic construct
  source 1..21: mol_type = other RNA
tttaaggtcagcggagcagct

SEQ ID NO: 13 - RNA, 21 nt, synthetic construct
  source 1..21: mol_type = other RNA
ttctccgaacgtgtcacgttt

SEQ ID NO: 14 - RNA, 21 nt, synthetic construct
  source 1..21: mol_type = other RNA
acgtgacacgttcggagaatt

SEQ ID NO: 15 - DNA, 20 bp, synthetic construct
  source 1..20: mol_type = other DNA
atgtcctggaagccattgag

SEQ ID NO: 16 - DNA, 22 bp, synthetic construct
  source 1..22: mol_type = other DNA
ccagttcattgaggctagagag

SEQ ID NO: 17 - DNA, 20 bp, synthetic construct
  source 1..20: mol_type = other DNA
ggcacctgtctcaagagttt

SEQ ID NO: 18 - DNA, 21 bp, synthetic construct
  source 1..21: mol_type = other DNA
agcccatccactggaataatg

SEQ ID NO: 19 - DNA, 22 bp, synthetic construct
  source 1..22: mol_type = other DNA
ctaggtttgatgcctccttctt

SEQ ID NO: 20 - DNA, 22 bp, synthetic construct
  source 1..22: mol_type = other DNA
gatggcttcataggtgacttcc

SEQ ID NO: 21 - DNA, 20 bp, synthetic construct
  source 1..20: mol_type = other DNA
gagccatggattgcactttc

SEQ ID NO: 22 - DNA, 21 bp, synthetic construct
  source 1..21: mol_type = other DNA
agcatagggtgggtcaaatag

SEQ ID NO: 23 - DNA, 20 bp, synthetic construct
  source 1..20: mol_type = other DNA
cctttgcctctccagttgaa

SEQ ID NO: 24 - DNA, 22 bp, synthetic construct
  source 1..22: mol_type = other DNA
gtacacaggtctcccttctttg

SEQ ID NO: 25 - DNA, 20 bp, synthetic construct
  source 1..20: mol_type = other DNA
tcctcggtgacttatcctgt

SEQ ID NO: 26 - DNA, 21 bp, synthetic construct
  source 1..21: mol_type = other DNA
gcgtggactccgtaatgatag

SEQ ID NO: 27 - DNA, 20 bp, synthetic construct
  source 1..20: mol_type = other DNA
caccaagcacaaacggaaag

SEQ ID NO: 28 - DNA, 20 bp, synthetic construct
  source 1..20: mol_type = other DNA
aggtgtggagcctagaagaa

SEQ ID NO: 29 - DNA, 20 bp, synthetic construct
  source 1..20: mol_type = other DNA
cagatcctgagcaagctgaa

SEQ ID NO: 30 - DNA, 22 bp, synthetic construct
  source 1..22: mol_type = other DNA
gtccctggtactgttgtagatg

SEQ ID NO: 31 - DNA, 20 bp, synthetic construct
  source 1..20: mol_type = other DNA
aggctgctgtaacgatgaag

SEQ ID NO: 32 - DNA, 20 bp, synthetic construct
  source 1..20: mol_type = other DNA
tctcctatgtgctggctttg

SEQ ID NO: 33 - DNA, 22 bp, synthetic construct
  source 1..22: mol_type = other DNA
gtagagacagctcgttgtactg

SEQ ID NO: 34 - DNA, 20 bp, synthetic construct
  source 1..20: mol_type = other DNA
gggatgccaccagacttaaa

SEQ ID NO: 35 - DNA, 20 bp, synthetic construct
  source 1..20: mol_type = other DNA
ccaagaaaggcaccgtagag

SEQ ID NO: 36 - DNA, 21 bp, synthetic construct
  source 1..21: mol_type = other DNA
cggaaagttggtgtccagtat

SEQ ID NO: 37 - DNA, 20 bp, synthetic construct
  source 1..20: mol_type = other DNA
tttcccggacaaacttccag

SEQ ID NO: 38 - DNA, 22 bp, synthetic construct
  source 1..22: mol_type = other DNA
gagcagattgaaggcagagtag

SEQ ID NO: 39 - DNA, 22 bp, synthetic construct
  source 1..22: mol_type = other DNA
gataagctggagtcacagaagg

SEQ ID NO: 40 - DNA, 22 bp, synthetic construct
  source 1..22: mol_type = other DNA
ttgccgagtagatctcaaagtg

SEQ ID NO: 41 - DNA, 22 bp, synthetic construct
  source 1..22: mol_type = other DNA
ctctgagaacctctgaaacgtc

SEQ ID NO: 42 - DNA, 21 bp, synthetic construct
  source 1..21: mol_type = other DNA
gaaactcagccgtctcttctt

SEQ ID NO: 43 - DNA, 21 bp, synthetic construct
  source 1..21: mol_type = other DNA
cagccttccttcttgggtatg

SEQ ID NO: 44 - DNA, 22 bp, synthetic construct
  source 1..22: mol_type = other DNA
ggcatagaggtctttacggatg

SEQ ID NO: 45 - RNA, 21 nt, synthetic construct
  source 1..21: mol_type = other RNA
gcagaaatgattgcactattt

SEQ ID NO: 46 - RNA, 21 nt, synthetic construct
  source 1..21: mol_type = other RNA
atagtgcaatcatttctgctt

SEQ ID NO: 47 - RNA, 21 nt, synthetic construct
  source 1..21: mol_type = other RNA
ctgctactcttcagcattatt

SEQ ID NO: 48 - RNA, 21 nt, synthetic construct
  source 1..21: mol_type = other RNA
taatgctgaagagtagcagtt

SEQ ID NO: 49 - RNA, 21 nt, synthetic construct
  source 1..21: mol_type = other RNA
cagtcccacttgtgtcaaatt

SEQ ID NO: 50 - RNA, 21 nt, synthetic construct
  source 1..21: mol_type = other RNA
tttgacacaagtgggactgtt

(19) United States
(12) Patent Application Publication
(10) Pub. No.: US 2026/0109987 A1
(43) Pub. Date: Apr. 23, 2026

(54) PREPARATION AND USE OF NUCLEIC ACID WITH ANTI-HAIR LOSS AND OIL CONTROL EFFECTS AND COMPOSITION INCLUDING NUCLEIC ACID

(71) Applicant: CANAAN PARTNERS INC., Newport Beach, CA (US)

(72) Inventors: Fang WANG, Newport Beach, CA (US); Haowei CHEN, Beijing (CN); Jie TAN, Beijing (CN); Jiaheng ZHANG, Shenzhen (CN); Nanxi XIANG, Chongqing (CN); Lijuan LIU, Chongqing (CN); Chunqiao ZHANG, Chongqing (CN); Chengyu WU, Shenzhen (CN); Mingqing ZHOU, Shenzhen (CN); Jichuan ZHANG, Shenzhen (CN)

(73) Assignee: CANAAN PARTNERS INC., Newport Beach, CA (US)

(21) Appl. No.: 19/363,685

(22) Filed: Oct. 21, 2025

(30) Foreign Application Priority Data
Oct. 21, 2024 (CN) 202411465074 .2

(51) Int. Cl. C12N 15/113; A61K 47/18; A61K 47/24; A61K 47/28; A61K 47/32; A61K 47/42; A61P 17/14

(52) U.S. Cl. CPC C12N 15/1138; A61K 47/186; A61K 47/24; A61K 47/28; A61K 47/32; A61K 47/42; A61P 17/14; C12N 2310/14

(57) ABSTRACT

A preparation and a use of a nucleic acid with anti-hair loss and oil control effects and a composition including the nucleic acid are provided. The small interfering RNA (siRNA) sequence has a significant silencing effect for the androgen receptor (AR) target gene. The composition prepared from an ionic liquid or a combination of an ionic liquid and a lipid exhibits a prominent delivery effect for water-soluble nucleic acid molecules. The composition can penetrate cells and enter cell nuclei to achieve an AR-silencing effect, and can provide significant regulatory effects for downstream target genes or proteins regulated by AR and comprehensive anti-hair loss and oil control effects. The composition exhibits a prominent transdermal permeation-promoting effect, and can permeate the epidermis layer and reach the dermis layer to exert remarkable anti-hair loss and oil control effects. In addition, the composition has a high antibacterial activity against Propionibacterium acnes.

### CROSS REFERENCE TO THE RELATED APPLICATIONS

This application is based upon and claims priority to Chinese Patent Application No. 202411465074.2, filed on Oct. 21, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBBJSR004-PKG_SequenceListing.xml, created on Jun. 11, 2025, and is 44,967 bytes in size.

### TECHNICAL FIELD

The present disclosure relates to the technical field of daily chemical products, and particularly relates to preparation and use of a nucleic acid with anti-hair loss and oil control effects and a composition including the nucleic acid.

### BACKGROUND

Hair loss is a widespread issue globally. According to the statistics of the World Health Organization, approximately 1.6 billion among the world population of 7.7 billion face the hair loss problem. That is, about 1 in every 4 individuals undergoes hair loss on average. Androgenetic alopecia (AGA), also known as seborrheic alopecia, is the most prevalent (accounting for 90% or more) among individuals with hair loss. Hair loss does not have a significant impact on the normal physiological functions of patients. However, hair loss can damage the appearance of patients, bring psychological burdens to patients, and even lead to anxiety and depression, thereby affecting the quality of life. Moreover, the relevant evidence shows that AGA may increase the risk of cardiovascular diseases and other diseases. Currently, the treatments for AGA mainly include medication, hair transplant surgery, low-level laser therapy (LLLT), hair regeneration technology, etc. Minoxidil and finasteride are the two drugs approved by the Food and Drug Administration (FDA) for treating AGA. These two therapeutic drugs may cause side effects including headaches, anxiety, hypertrichosis, irritation, contact dermatitis, memory decline, and sexual desire suppression. Minoxidil has been associated with reported fetal teratogenicity and thus is not suitable for pregnant and breastfeeding women. Additionally, after the administration of minoxidil is discontinued, hair loss often recurs, making long-term sustained therapy necessary. The hair transplant is invasive and expensive and has drawbacks such as long operation time, postoperative pain, and risk of infection. LLLT can prolong the anagen phase of hair follicles and increase the number of hair follicles at the anagen phase by stimulating the transition of catagen and telogen hair follicles into anagen hair follicles and demonstrates efficacy in patients with mild-to-moderate AGA. However, LLLT is associated with side effects including edema, erythema, dandruff, local pain, pruritus, erosion, and hair shaft damage.

Nucleic acid-based pharmaceutical preparations, as a popular field of preparation development, provide a new idea and method for the development of vaccines and the treatment of tumors, metabolic disorders, and genetic diseases. For example, RNA interference (RNAi) technology is currently a hot and challenging topic in the field of gene therapy drugs. RNAi refers to the biological process of highly specific degradation of homologous mRNA induced by double-stranded RNA. The RNAi technology includes small interfering RNA (siRNA) and asymmetric small interfering RNA (asiRNA) technologies. Because the siRNA technology may activate non-specific innate immune responses, the asiRNA technology is developed as an alternative and complementary strategy to overcome the nonspecific effect. According to the principle of AGA, the androgen receptor (AR) plays a key role in hair loss. If siRNA or asiRNA targeting the AR gene is designed to inhibit the expression of AR, the hair loss can be prevented, and even the hair growth can be promoted. Compared with the prior techniques, the RNAi technology demonstrates a silencing effect for a specific target gene and has advantages such as high efficiency, specificity, designability, instant onset, and easy detection of effects. However, nucleic acids exposed under natural conditions exhibit poor stability, are susceptible to contamination by external microorganisms, and are easily degraded by nucleases during in vitro or in vivo circulation. Moreover, the water solubility and large molecular structures of nucleic acids result in poor permeability and low bioavailability. Therefore, to make full use of the RNAi technology and achieve the therapeutic effects of nucleic acid-based drugs, it is necessary to design appropriate RNA delivery systems to enable effective silencing and targeted and efficient hair loss prevention.

The targets currently reported for nucleic acids or compositions thereof associated with hair loss prevention/hair strengthening include: receptor-interacting protein (RIP) kinases, 3-oxo-5-α-steroid 4-dehydrogenase 1 (SRD5A1), 3-oxo-5-α-steroid 4-dehydrogenase 2 (SRD5A2), AR, and fibroblast growth factor 5 (FGF5). The above outcomes merely indicate that nucleic acids have respective anti-hair loss effects inherently, but it fails to achieve the transformation of nucleic acids into practical production applications. It is well known that nucleic acid-based drugs are highly sensitive and prone to degradation by nucleases in the environment. Double-stranded nucleic acids typically have a molecular weight of 10,000 Da or more and exhibit strong hydrophilicity, hindering the transdermal delivery. Consequently, the transformation of nucleic acid raw materials into nucleic acid-based preparations is very challenging. The current mainstream delivery systems for nucleic acid-based pharmaceutical preparations are specialized cationic liposome delivery systems, which are relatively expensive. In addition, the production of liposome delivery systems often involves the use of toxic organic solvents. The residual solvent and the complex preparation process are unfavorable for the popularization and large-scale production.

### SUMMARY

An objective of the present disclosure is to overcome the deficiencies of the prior art and to provide preparation and use of a nucleic acid with anti-hair loss and oil control effects and a composition including the nucleic acid.

To achieve the above objective, the present disclosure adopts the following technical solutions:

In a first aspect, the present disclosure provides a nucleic acid with anti-hair loss and oil control effects, where the nucleic acid is siRNA targeting AR, and sequences of double strands of the nucleic acid are at least one group of the following or are produced through a chemical modification based on at least one group of the following:
(1) sequences shown in SEQ ID NO: 1 and SEQ ID NO: 2 (si-AR-1), respectively;
(2) sequences shown in SEQ ID NO: 3 and SEQ ID NO: 4 (si-AR-2), respectively; and
(3) sequences shown in SEQ ID NO: 5 and SEQ ID NO: 6 (si-AR-3), respectively.

The siRNA sequence designed in the present disclosure and a sequence produced by chemically modifying the siRNA sequence have a significant silencing effect for the AR target gene.

As a preferred embodiment of the nucleic acid in the present disclosure, the sequences of the double strands of the nucleic acid are shown in SEQ ID NO: 3 and SEQ ID NO: 4 (si-AR-2) or shown in SEQ ID NO: 5 and SEQ ID NO: 6 (si-AR-3), respectively. Nucleic acids with the corresponding sequences have a significant silencing effect for the AR target gene.

As a preferred embodiment of the nucleic acid in the present disclosure, the sequences of the double strands of the nucleic acid are shown in SEQ ID NO: 5 and SEQ ID NO: 6 (si-AR-3), respectively. A nucleic acid with the corresponding sequences has a significant silencing effect for the AR target gene.

As a preferred embodiment of the nucleic acid in the present disclosure, the chemical modification includes at least one of the following:
a, a modification of substituting oxygen in a sugar structure of a nucleotide with sulfur;
b, a modification of converting a nucleotide bond into a thiophosphate bond, a boranophosphate bond, or a methylphosphate bond;
c, a modification of producing a peptide nucleic acid (PNA), a locked nucleic acid (LNA), or an unlocked nucleic acid (UNA); and
d, a modification of combining cholesterol or a cell-penetrating peptide.

As a preferred embodiment of the nucleic acid in the present disclosure, the chemical modification is at least one of a modification of converting a nucleotide bond into a thiophosphate bond or a modification of combining cholesterol.

As a preferred embodiment of the nucleic acid in the present disclosure, the nucleic acid has sequences of Chol-G*C*A*G*A*A*A*U*G*A*U*U*G*C*A*C*U*A*U*T*T* shown in SEQ ID NO: 45 and A*U*A*G*U*G*C*A*A*U*C*A*U*U*U*C*U*G*C*T*T* shown in SEQ ID NO: sequences 46 (PC-AR-1), of Chol-C*U*G*C*U*A*C*U*C*U*U*C*A*G*C*A*U*U*A*T*T* shown in SEQ ID NO: 47 and U*A*A*U*G*C*U*G*A*A*G*A*G*U*A*G*C*A*G*T*T* shown in SEQ ID NO: 48 (PC-AR-2), or sequences of Chol-C*A*G*U*C*C*C*A*C*U*U*G*U*G*U*C*A*A*A*T*T* shown in SEQ ID NO: 49 and U*U*U*G*A*C*A*C*A*A*G*U*G*G*G*A*C*U*G*T*T* shown in SEQ ID NO: 50 (PC-AR-3), where Chol represents cholesterol and * represents a thiophosphate backbone. The modified siRNA sequence designed in the present disclosure has a significant silencing effect for the AR target gene.

As a preferred embodiment of the nucleic acid in the present disclosure, the nucleic acid has sequences of Chol-G*C*A*G*A*A*A*U*G*A*U*U*G*C*A*C*U*A*U*T*T* shown in SEQ ID NO: 45 and A*U*A*G*U*G*C*A*A*U*C*A*U*U*U*C*U*G*C*T*T* shown in SEQ ID NO: 46 (PC-AR-1) or sequences of Chol-C*U*G*C*U*A*C*U*C*U*U*C*A*G*C*A*U*U*A*T*T* shown in SEQ ID NO: 47 and U*A*A*U*G*C*U*G*A*A*G*A*G*U*A*G*C*A*G*T*T* shown in SEQ ID NO: 48 (PC-AR-2), where Chol represents cholesterol and * represents a thiophosphate backbone. The modified siRNA sequence has a significant silencing effect for the AR target gene.

As a preferred embodiment of the nucleic acid in the present disclosure, the nucleic acid has sequences of Chol-G*C*A*G*A*A*A*U*G*A*U*U*G*C*A*C*U*A*U*T*T* shown in SEQ ID NO: 45 and A*U*A*G*U*G*C*A*A*U*C*A*U*U*U*C*U*G*C*T*T* shown in SEQ ID NO: 46 (PC-AR-1), where Chol represents cholesterol and * represents a thiophosphate backbone. The modified siRNA sequence has a significant silencing effect for the AR target gene.

In a second aspect, the present disclosure provides a use of the nucleic acid in preparation of a composition with anti-hair loss and oil control effects.

In a third aspect, the present disclosure provides a composition, including the following components in weight percentages: 0.00008% to 0.012% of the nucleic acid, 0.0002% to 5% of an ionic liquid, 0.0004% to 0.12% of a polymer, and the balance of a solvent, where the ionic liquid has a structural formula shown in a formula (I):

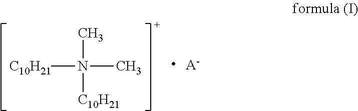

where A⁻ represents an organic or inorganic counter ion. Preferably, the A⁻ in the formula (I) includes HCOO⁻, CH₃COO⁻, CH₃CH₂COO⁻, CH₃CH₂CH₂COO⁻, Cl⁻, Br⁻, or NO₃⁻.

As a preferred embodiment of the composition in the present disclosure, the polymer includes at least one of polyethyleneimine, polyquaternium-7 (dimethyl diallyl ammonium chloride-acrylamide copolymer with a viscosity of 8,000 cps to 15,000 cps at a content of 10%), polyarginine, or polylysine (with a molecular weight of 3,600 to 4,300). The polyethyleneimine is branched polyethyleneimine (BPEI) or linear polyethyleneimine (LPEI), and has an average molecular weight of 3,000 to 100,000. The polyarginine includes 6, 7, 9, or 11 polymerization units, or has a molecular weight of less than 2,000. The solvent includes water, an alcohol, or an ether. The water includes ultrapure water or diethyl pyrocarbonate (DEPC)-treated water. The alcohol includes 1,2-pentanediol, 1,3-propanediol, 1,2-butanediol, ethylene glycol, glycerin, or 1,2-hexanediol. The ether is isosorbide dimethyl ether.

As a preferred embodiment of the composition in the present disclosure, the ionic liquid includes at least one of didecyldimethylammonium chloride, didecyldimethylammonium bromide, didecyldimethylammonium formate, or didecyldimethylammonium nitrate, and has a melting point of lower than 100° C. The solvent includes ultrapure water, 1,2-pentanediol, isosorbide dimethyl ether, glycerin, or 1,2-hexanediol.

As a preferred embodiment of the composition in the present disclosure, the composition further includes a lipid and/or a polypeptide.

As a preferred embodiment of the composition in the present disclosure, in a weight percentage, the composition further includes 0.011% to 0.33% of the lipid.

As a preferred embodiment of the composition in the present disclosure, in a weight percentage, the composition further includes 0.001% to 0.22% of the polypeptide.

As a preferred embodiment of the composition in the present disclosure, in weight percentages, the composition further includes 0.011% to 0.33% of the lipid and 0.001% to 0.22% of the polypeptide.

As a preferred embodiment of the composition in the present disclosure, the lipid includes at least one of hydrogenated lecithin, soy lecithin, phosphatidylethanolamine, dipalmitoylphosphatidylcholine, distearoylphosphatidylcholine, or cholesterol, and the polypeptide includes at least one of acetyl tetrapeptide-3, acetyl dipeptide-1 cetyl ester, biotinoyl tripeptide-1, or oligopeptide-54.

As a preferred embodiment of the composition in the present disclosure, the lipid includes at least one of hydrogenated lecithin, soy lecithin, or cholesterol.

As a preferred embodiment of the composition in the present disclosure, the soy lecithin includes PC90, PC50, or PC70.

In a fourth aspect, the present disclosure provides a preparation method of the composition, where the preparation method is any one of the following:

A, including the following steps:
S1, dissolving the polymer and the nucleic acid with a part of the solvent (a part of the total solvent) separately to produce a polymer solution and a nucleic acid solution, and refrigerating the polymer solution and the nucleic acid solution at 0° C. to 10° C.;
S2, mixing the ionic liquid with a remaining part of the solvent (a sum of a remaining part of the solvent and the part of the solvent in the S1 is the total solvent), and allowing complete dissolution to produce a mixture; and refrigerating the mixture at 0° C. to 10° C.; and
S3, adding a refrigerated polymer solution obtained in the S1 to a refrigerated mixture obtained in the S2, and thoroughly mixing; and adding a refrigerated nucleic acid solution obtained in the S1, and incubating for 1 h to 4 h to produce the composition; B, including the following steps:
S1, dissolving the polymer and the nucleic acid with a part of the solvent (a part of the total solvent) separately to produce a polymer solution and a nucleic acid solution, and refrigerating the polymer solution and the nucleic acid solution at 0° C. to 10° C.;
S2, mixing the lipid, the ionic liquid, and a part of the solvent (a part of the total solvent), and allowing complete dissolution at 40° C. to 60° C. to produce a mixture; adding a remaining part of the solvent (a sum of a remaining part of the solvent, the part of the solvent in the S1, and the part of the solvent in the S2 is the total solvent) at 40° C. to 60° C. to the mixture, and stirring for 5 min to 10 min; conducting high-speed shearing at 3,000 r/min to 5,000 r/min for 1 min to 3 min, and conducting high-pressure homogenization 2 times to 6 times at 200 bar to 600 bar to produce a homogeneous mixture; and refrigerating the homogeneous mixture at 0° C. to 10° C.; and
S3, adding a refrigerated polymer solution obtained in the S1 to a refrigerated homogeneous mixture obtained in the S2, and thoroughly mixing; and adding a refrigerated nucleic acid solution obtained in the S1, and incubating for 1 h to 4 h to produce the composition; and
C, including the following steps:
S1, dissolving the polymer, the nucleic acid, and the polypeptide with a part of the solvent (a part of the total solvent) separately to produce a polymer solution, a nucleic acid solution, and a polypeptide solution, and refrigerating the polymer solution, the nucleic acid solution, and the polypeptide solution at 0° C. to 10° C.;
S2, mixing the lipid, the ionic liquid, and a part of the solvent (a part of the total solvent), and allowing complete dissolution at 40° C. to 60° C. to produce a mixture; adding a remaining part of the solvent (a sum of a remaining part of the solvent, the part of the solvent in the S1, and the part of the solvent in the S2 is the total solvent) at 40° C. to 60° C. to the mixture, and stirring for 5 min to 10 min; conducting high-speed shearing at 3,000 r/min to 5,000 r/min for 1 min to 3 min, and conducting high-pressure homogenization 2 times to 6 times at 200 bar to 600 bar to produce a homogeneous mixture; and refrigerating the homogeneous mixture at 0° C. to 10° C.; and
S3, adding a refrigerated polymer solution obtained in the SI to a refrigerated homogeneous mixture obtained in the S2, and thoroughly mixing; adding a refrigerated polypeptide solution obtained in the S1, and thoroughly mixing; and adding a refrigerated nucleic acid solution obtained in the S1, and incubating for 1 h to 4 h to produce the composition.

As a preferred embodiment of the preparation method in the present disclosure, the refrigerating is conducted at 2° C. to 8° C.

In a fifth aspect, the present disclosure provides a use of the composition in preparation of a product with anti-hair loss and oil control effects.

As a preferred embodiment of the use in the present disclosure, the product includes an anti-hair loss gel formulation, an oil-control and anti-acne serum formulation, or an anti-hair loss and hair-strengthening soluble microneedle formulation.

In a sixth aspect, the present disclosure provides a product with anti-hair loss and oil control effects, including the composition.

Compared with the prior art, the present disclosure has the following beneficial effects:

The siRNA sequence designed in the present disclosure has a significant silencing effect for the AR target gene. The composition prepared from an ionic liquid or a combination of an ionic liquid and a lipid in the present disclosure exhibits a prominent delivery effect for water-soluble nucleic acid molecules. The composition can penetrate cells and enter cell nuclei to achieve an AR-silencing effect, and can provide significant regulatory effects for downstream target genes or proteins regulated by AR and comprehensive anti-hair loss and oil control effects. The composition of the present disclosure exhibits a prominent transdermal permeation-promoting effect, and can permeate the epidermis layer and reach the dermis layer to exert remarkable anti-hair loss and oil control effects. In addition, the composition of the present disclosure has a high antibacterial activity against Propionibacterium acnes. The composition of the present disclosure is a nucleic acid raw material with anti-hair loss, oil control, and anti-acne triple effects, which expands new applications for AR-targeted nucleic acids.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show the relative expression levels of an AR protein in cells transfected with different nucleic acids in Example 1 of the present disclosure, where FIG. 1A shows the quantitative analysis results and FIG. 1B shows the western blot results;

FIG. 2 shows the relative expression levels of an AR gene in cells transfected with different nucleic acids in Example 1 of the present disclosure;

FIGS. 3A-3B show the appearance of compositions 1 to 4 and 17 to 22 of the present disclosure and compositions of Comparative Examples 2 and 3, where FIG. 3A shows the compositions 1 to 4 and the compositions in Comparative Examples 2 and 3 and FIG. 3B shows the compositions 17 to 22;

FIG. 4 shows the zeta potential measurement results of a composition 32 of the present disclosure;

FIG. 5 shows the zeta potential measurement results of a composition 35 of the present disclosure;

FIG. 6 shows the particle size distribution results of a composition 14 of the present disclosure before the addition of a nucleic acid;

FIG. 7 shows the particle size distribution results of the composition 14 of the present disclosure after the addition of the nucleic acid;

FIGS. 8A-8B show the transmission electron microscopy (TEM) images of the composition 14 of the present disclosure before and after the addition of the nucleic acid, where FIG. 8A shows the composition 14 without the nucleic acid and FIG. 8B shows the composition 14 with the nucleic acid;

FIGS. 9A-9B show the gel electrophoresis results of the composition 14 of the present disclosure that has been stored in a −15° C. environment for 3 months, where FIG. 9A is for si-AR-4 and FIG. 9B is for the composition 14;

FIGS. 10A-10H show the fluorescent transdermal results of a nucleic acid-containing phosphate buffered saline (PBS) solution and compositions 1, 14, and 29 in the present disclosure at 4 h and 16 h, where FIG. 10A shows a fluorescent transdermal result of the nucleic acid-containing PBS solution at 4 h, FIG. 10B shows a fluorescent transdermal result of the nucleic acid-containing PBS solution at 16 h, FIG. 10C shows a fluorescent transdermal result of the composition 1 at 4 h, FIG. 10D shows a fluorescent transdermal result of the composition 1 at 16 h, FIG. 10E shows a fluorescent transdermal result of the composition 2 at 4 h, FIG. 10F shows a fluorescent transdermal result of the composition 2 at 16 h, FIG. 10G shows a fluorescent transdermal result of the composition 3 at 4 h, and FIG. 10H shows a fluorescent transdermal result of the composition 3 at 16 h;

FIGS. 11A-11I show the fluorescence results of cells transfected with PBS and compositions 14 and 29 in the present disclosure, where FIG. 11A shows a fluorescence result of FAM-asi-AR-3 for PBS, FIG. 11B shows a fluorescence result of FAM-asi-AR-3 for the composition 14, FIG. 11C shows a fluorescence result of FAM-asi-AR-3 for the composition 29, FIG. 11D shows a fluorescence result of 4′,6-diamidino-2-phenylindole (DAPI) for PBS, FIG. 11E shows a fluorescence result of DAPI for the composition 14, FIG. 11F shows a fluorescence result of DAPI for the composition 29, FIG. 11G shows a fluorescence result of Merge for PBS, FIG. 11H shows a fluorescence result of Merge for the composition 14, and FIG. 11I shows a fluorescence result of Merge for the composition 29;

FIGS. 12A-12B show the relative expression levels of the AR protein in cells transfected with compositions 1, 14, 29, and 35 of the present disclosure, where FIG. 12A shows the quantitative analysis results and FIG. 12B shows the western blot results;

FIG. 13 shows the expression of vascular endothelial growth factor (VEGF) corresponding to the compositions 1, 14, 29, and 35 of the present disclosure;

FIGS. 14A-14I show the influence of the compositions 1, 14, and 29 of the present disclosure on the relative expression levels of the target protein AR and the related proteins DDK-1, TGF-β2, BMP4, IL-1α, IL-8, TNF-α, β-catenin, and MMP3, where FIG. 14A is for AR, FIG. 14B is for DDK-1, FIG. 14C is for TGF-β2, FIG. 14D is for BMP4, FIG. 14E is for IL-1α, FIG. 14F is for IL-8, FIG. 14G is for TNF-α, FIG. 14H is for β-catenin, and FIG. 14I is for MMP3;

FIG. 15 shows the western blot results of influence of the compositions 1, 14, and 29 of the present disclosure on the target protein AR and the related proteins IL-1α, IL-8, TNF-α, DKK-1, TGF-β2, BMP4, β-catenin, and MMP3;

FIGS. 16A-16B show the influence of the compositions 1, 14, and 29 of the present disclosure on lipids of SZ95 cells, where FIG. 16A shows the Nile red staining results and FIG. 16B shows the semi-quantitative analysis results of relative lipid contents; and

FIGS. 17A-17F show the influence of the compositions 1, 14, and 29 of the present disclosure on AR and lipid-related proteins FASN, SREBP1c, CD36, and PPARa, where FIGS. 17A-17E show the relative expression levels of the proteins and FIG. 17F shows the western blot results.

### DETAILED DESCRIPTION OF THE EMBODIMENTS

In order to well illustrate the objectives, technical solutions, and advantages of the present disclosure, the present disclosure will be further described below in conjunction with specific examples.

The materials, reagents, etc. adopted in the examples all are commercially available, unless otherwise specified.

Polyarginine, polylysine, and polyethyleneimine with different molecular weights and paraformaldehyde: Macklin Biochemical Technology Co., Ltd. Oligopeptide-54, biotinoyl tripeptide-1, and acetyl dipeptide-1 cetyl ester: Shenzhen Readline Biotech Co., Ltd. Acetyl tetrapeptide-3: Chengdu YoungShe Chemical Co., Ltd. Hydrogenated lecithin and soy lecithin: Shanghai Haoyun Trade Co., Ltd. Didecyldimethylammonium chloride, didecyldimethylammonium bromide, didecyldimethylammonium formate, or didecyldimethylammonium nitrate: China Research Institute of Daily Chemical Industry. Hair follicle dermal papilla cells (HFDPCs): Wuhan Pricella Biotechnology Co., Ltd. Opti-MEM, Lipo6000, NanoDrop spectrophotometer, fluorescent quantitative polymerase chain reaction (PCR) 8-tube strip, and cDNA Synthesis Kit: Thermo Scientific. Electrophoresis apparatus, transfer electrophoresis tank, and vertical electrophoresis tank: CAVOY. Microplate reader: TECAN. Low-temperature centrifuge: Eppendorf. Gel imaging system: GE. Benchtop high-speed refrigerated microcentrifuge: DragonLab. Fluorescent quantitative PCR instrument: Roche. 2×KAPA qPCR Master Mix: Kapa Biosystems. Primers: GenePharma. Did: Beyotime Biotech Inc., Shanghai. DAPI: Beijing Solarbio Science & Technology Co., Ltd. Laser scanning confocal microscope: ZEISS, Germany.

Primer sequences adopted in the present disclosure are shown in Table 1.

TABLE 1

| Name | Sense strand 5′-3′ | Antisense strand 5′-3′ |
|---|---|---|
| Primer 1 | ATGTCCTGGAAGCCATTGAG (SEQ | CCAGTTCATTGAGGCTAGAGAG |
| (AR Homo sapien) | ID NO: 15) | (SEQ ID NO: 16) |
| Primer 2 | GGCACCTGTCTCAAGAGTTT (SEQ | AGCCCATCCACTGGAATAATG |
| (AR Mus musculus) | ID NO: 17) | (SEQ ID NO: 18) |
| Primer 3 | CTAGGTTTGATGCCTCCTTCTT | GATGGCTTCATAGGTGACTTCC |
| (fatty acid synthase (FASN)) | (SEQ ID NO: 19) | (SEQ ID NO: 20) |
| Primer 4 | GAGCCATGGATTGCACTTTC (SEQ | AGCATAGGGTGGGTCAAATAG |
| (SREBF1) | ID NO: 21) | (SEQ ID NO: 22) |
| Primer 5 | CCTTTGCCTCTCCAGTTGAA (SEQ | GTACACAGGTCTCCCTTCTTTG |
| (CD36) | ID NO: 23) | (SEQ ID NO: 24) |
| Primer 6 | TCCTCGGTGACTTATCCTGT (SEQ | GCGTGGACTCCGTAATGATAG |
| (PPARa) | ID NO: 25) | (SEQ ID NO: 26) |
| Primer 7 | CACCAAGCACAAACGGAAAG | AGGTGTGGAGCCTAGAAGAA |
| (DKK1) | (SEQ ID NO: 27) | (SEQ ID NO: 28) |
| Primer 8 | CAGATCCTGAGCAAGCTGAA (SEQ | GTCCCTGGTACTGTTGTAGATG |
| (TGFβ2) | ID NO: 29) | (SEQ ID NO: 30) |
| Primer 9 | AGGCTGCTGTAACGATGAAG (SEQ | TCTCCTATGTGCTGGCTTTG |
| (VEGF) | ID NO: 31) | (SEQ ID NO: 32) |
| Primer 10 | GTAGAGACAGCTCGTTGTACTG | GGGATGCCACCAGACTTAAA |
| (β-catenin (Ctnnbl)) | (SEQ ID NO: 33) | (SEQ ID NO: 34) |
| Primer 11 | CCAAGAAAGGCACCGTAGAG (SEQ | CGGAAAGTTGGTGTCCAGTAT |
| (Col17A1) | ID NO: 35) | (SEQ ID NO: 36) |
| Primer 12 | TTTCCCGGACAAACTTCCAG (SEQ | GAGCAGATTGAAGGCAGAGTAG |
| (TCF1 (Tcf7)) | ID NO: 37) | (SEQ ID NO: 38) |
| Primer 13 | GATAAGCTGGAGTCACAGAAGG | TTGCCGAGTAGATCTCAAAGTG |
| (IL-6) | (SEQ ID NO: 39) | (SEQ ID NO: 40) |
| Primer 14 | CTCTGAGAACCTCTGAAACGTC | GAAACTCAGCCGTCTCTTCTT |
| (IL-1α) | (SEQ ID NO: 41) | (SEQ ID NO: 42) |
| Primer 15 | CAGCCTTCCTTCTTGGGTATG (SEQ | GGCATAGAGGTCTTTACGGATG |
| (β-actin (Actb)) | ID NO: 43) | (SEQ ID NO: 44) |

### Example 1

### 1. Design of Nucleic Acids

The human AR gene was queried from the National Center for Biotechnology Information (NCBI) gene database. siRNAs and asiRNAs targeting the AR gene were designed in the coding sequence (CDS) region according to the siRNA screening principle, and synthesized by Suzhou GenePharma Co., Ltd. Sequences of the siRNAs and asiRNAs of the present disclosure are shown in Table 2 below, where si-NC represents a negative control (NC) without a silencing effect.

TABLE 2

| Name | Sense strand 5′-3′ | Antisense strand 5′-3′ |
|---|---|---|
| si-AR-1 | GCAGAAAUGAUUGCACUAUTT (SEQ ID NO: 1) | AUAGUGCAAUCAUUUCUGCTT (SEQ ID NO: 2) |
| si-AR-2 | CUGCUACUCUUCAGCAUUATT (SEQ ID NO: 3) | UAAUGCUGAAGAGUAGCAGTT (SEQ ID NO: 4) |
| si-AR-3 | CAGUCCCACUUGUGUCAAATT (SEQ ID NO: 5) | UUUGACACAAGUGGGACUGIT (SEQ ID NO: 6) |
| PC-AR-1 | Chol- | A*U*A*G*U*G*C*A*A*U*C*A*U*U*U*C*U*G*C |
| | G*C*A*G*A*A*A*U*G*A*U*U*G*C*A*C*U*A*U* | *T*T* (SEQ ID NO: 46) |
| | T*T* (SEQ ID NO: 45) | |
| PC-AR-2 | Chol- | U*A*A*U*G*C*U*G*A*A*G*A*G*U*A*G*C*A* |
| | C*U*G*C*U*A*C*U*C*U*U*C*A*G*C*A*U*U*A*T | G*T*T* (SEQ ID NO: 48) |
| | *T* (SEQ ID NO: 47) | |
| PC-AR-3 | Chol- | U*U*U*G*A*C*A*C*A*A*G*U*G*G*G*A*C*U* |
| | C*A*G*U*C*C*C*A*C*U*U*G*U*G*U*C*A*A*A*T | G*T*T* (SEQ ID NO: 50) |
| | *T* (SEQ ID NO: 49) | |
| asi-AR-1 | CUUUUGACCUGCUAAU (SEQ ID NO: 7) | AUUAGCAGGUCAAAAGUGAAC (SEQ ID NO: 8) |
| asi-AR-2 | UCAAGCCCAUCUAUUU (SEQ ID NO: 9) | AAAUAGAUGGGCUUGACUUUC (SEQ ID NO: 10) |
| asi-AR-3 | CUCCGCUGACCUUAAA (SEQ ID NO: 11) | UUUAAGGUCAGCGGAGCAGCU (SEQ ID NO: 12) |
| si-NC | UUCUCCGAACGUGUCACGUTT (SEQ ID NO: 13) | ACGUGACACGUUCGGAGAATT (SEQ ID NO: 14) |

Notes: “*” indicates a modification of a thiophosphate backbone. Explanation of SEQ ID NOS: 1-14 in the sequence listing of the specification: According to the editing rules of the WIPO Sequence software, a nucleotide sequence must only include the symbols listed in “Part 1 of Annex I of WIPO ST.26”. The base “t” represents “u” in an RNA sequence. Thus, SEQ ID NOS: 1-14 in the specification of the present disclosure are essentially the same as SEQ ID NOS: 1-14 in the sequence listing.

### 2. Testing of Effects of the Nucleic Acids

To determine whether a designed nucleic acid had an AR gene-targeted silencing effect, the commercial transfection reagent Lipo6000 was used to conduct a transfection test according to instructions. Human HFDPC was selected as a cell line. HFDPCs of generations 4 to 8 were selected for the transfection test.

### (1) Transfection Operation Steps:

1) siRNA or asiRNA was centrifuged briefly before use, then prepared into a 20 μM stock solution with DEPC-treated water, and then dispensed and stored.
2) Passage of HFDPCs: 24 h before transfection, 2×10⁵ cells were inoculated (in a 12-well plate) and cultured until a cell confluency was 30% to 50%.
3) Transfection: A solution A (1.25 μL of siRNA or asiRNA+250 μL of Opti-MEM) was gently pipetted up and down 3 to 5 times and allowed to stand for 5 min. A solution B (1 μL of a transfection reagent+250 μL of Opti-MEM) was gently pipetted up and down 3 to 5 times and allowed to stand for 5 min. The solution A and the solution B were mixed, gently pipetted up and down 3 to 5 times, allowed to stand for 10 min, and then added to the plate for transfection (a final nucleic acid concentration was 50 nM).
4) Cells were cultured in a 37° C. and 5% CO2 incubator for 6 h (fluorescence detection, FAM-labeled siRNA or asiRNA). Then the original medium was replaced with a fetal bovine serum (FBS)-free 1640 medium, and cells were further cultured for 42 h. The gene and protein detection was conducted.

### (2) Protein Detection

The protein detection was conducted by Western blot. Detection steps were as follows:
1) The transfected cells were cultured in a 37° C. and 5% CO2 incubator for 48 h. A cell pellet was then collected, an RIPA lysis buffer was added, and lysis was allowed at 4° C. for 30 min. A resulting lysate was centrifuged at 12,000 rpm for 10 min, and a resulting supernatant was collected and subjected to total protein extraction.
2) A protein concentration was determined by the bicinchoninic acid (BCA) assay.
3) 40 μg of a protein sample was taken and thoroughly mixed with 5× loading buffer (volume ratio: 4:1). Then proteins were separated by sodium dodecyl sulfate-polyacrylamide gel electrophoresis (SDS-PAGE).
4) The proteins were electrotransferred to a polyvinylidene fluoride (PVDF) membrane, and blocking was conducted with a 5% skimmed milk powder for 1 h.
5) A primary antibody was added, and incubation was conducted overnight at 4° C. Rinsing was conducted with TBST 3 times for 10 min each time.
6) On the next day, a horseradish peroxidase-labeled secondary antibody was added, and incubation was conducted for 1 h at room temperature of 37° C. Rinsing was conducted with TBST 3 times for 10 min each time.
7) Visualization was allowed based on the chemiluminescence of an enhanced chemiluminescence (ECL) substrate. An integral absorbance value of a band was quantitatively analyzed with the Quantity One software. An expression level of a protein to be tested was reflected with an integral absorbance ratio.

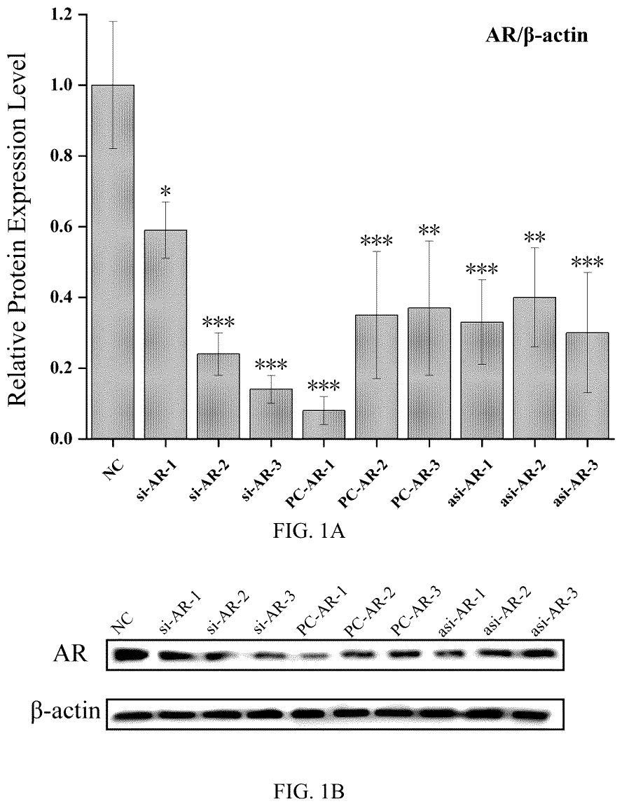

Relative expression levels of the AR protein in cells transfected with different nucleic acids are shown in FIGS. 1A-1B. FIG. 1A shows the quantitative analysis results, FIG. 1B shows the western blot results, *P<0.05, **P<0.01, and ***P<0.001. The results show that, compared with the NC, the siRNAs (si-AR-1, si-AR-2, and si-AR-3), modified siRNAs (PC-AR-1, PC-AR-2, and PC-AR-3), and asiRNAs (asi-AR-1, asi-AR-2, and asi-AR-3) of the present disclosure all have significant silencing effects for the target protein AR. The si-AR-2, si-AR-3, and PC-AR-1 exhibit the most significant silencing effects. The modified sequences PC-AR-2 and PC-AR-3 corresponding to the si-AR-2 and si-AR-3 do not have a stronger silencing effect for the AR protein than the si-AR-2 and si-AR-3, indicating that a modification for the sequences affects a silencing effect to some extent, but does not always lead to a positive effect. For siRNAs with strong silencing effects, a modification may actually reduce the silencing effect for the target protein.

### (3) Gene Detection

The gene detection was conducted by reverse transcription-polymerase chain reaction (RT-PCR), including the following steps:
1) The transfected cells were cultured in a 37° C. and 5% CO₂ incubator for 48 h, and the total RNA was then extracted from cells by the TRIZOL method.
2) According to an RNA concentration, 1 μg of an RNA template and 1 μL of an oligo (dT) 18 primer were taken, and DEPC-treated water was added to 12 μL. A reaction was allowed in a constant-temperature metal bath at 65° C. for 5 min, and a resulting reaction system was then immediately pre-cooled on ice.
3) 4 μL of 5× Reaction buffer, 1 μL of Ribolock Rnase inhibitor, 2 μL of 10 mM dNTP mix, and 1 μL of RevertAid M-Mulv (200 U/μL) were mixed and added to a pre-cooled system obtained in the step 2). A reaction was allowed in a constant-temperature metal bath at 42° C. for 60 min, and then a reaction was allowed at 70° C. for 5 min. A cDNA product was cryopreserved at −80° C. for later use.
4) 0.4 μL of the cDNA (50 ng/μL) synthesized in the step 3) was mixed with 10 μL of 2×KAPA qPCR Master Mix Universal, 0.4 μL of 10 μM Forward primer, 0.4 μL of 10 μM Reverse primer, and 8.8 μL of DEPC-treated water.
5) Amplification testing was conducted on a fluorescent quantitative PCR instrument (because a product was less than 300 bp, a two-step method was adopted for amplification) under the following reaction conditions: 95° C./3 min, 1 cycle; and 95° C./30 s, 60° C./30 s, 40 cycles in total. According to a Ct value, a relative expression level of mRNA was expressed by 2^{-ΔΔCt}.

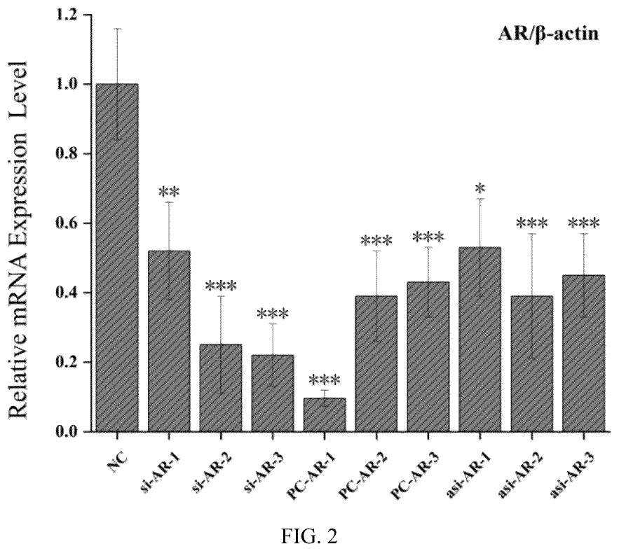

Relative expression levels of the AR gene in cells transfected with different nucleic acids are shown in FIG. 2. In FIG. 2, *P<0.05, **P<0.01, and ***P<0.001. The results show that, compared with the NC, the siRNAs (si-AR-1, si-AR-2, and si-AR-3), modified siRNAs (PC-AR-1, PC-AR-2, and PC-AR-3), and asiRNAs (asi-AR-1, asi-AR-2, and asi-AR-3) of the present disclosure all have significant silencing effects for the target protein AR. The si-AR-2, si-AR-3, and PC-AR-1 exhibit the most significant silencing effects. The modified sequences PC-AR-2 and PC-AR-3 corresponding to the si-AR-2 and si-AR-3 do not have a stronger silencing effect for the AR gene than the si-AR-2 and si-AR-3, indicating that modified siRNA corresponding to siRNA with a prominent silencing effect does not necessarily also exhibit a prominent silencing effect.

In summary, it can be seen that the siRNAs and asiRNAs designed for human HFDPCs and the modified siRNAs in which a 5′ terminus of a sense strand is modified with cholesterol or a thiophosphate backbone is modified in the present disclosure have significant inhibitory effects for the AR target at both protein and gene levels. The si-AR-2, si-AR-3, and PC-AR-1 exhibit the most significant inhibitory effects for the protein and gene of the AR target.

### Example 2

A composition was prepared with the nucleic acid in Example 1. The composition of the present disclosure includes the nucleic acid, an ionic liquid, a polymer, and a solvent. In weight percentages, the composition includes 0.00008% to 0.012% of the nucleic acid, 0.0002% to 5% of the ionic liquid, 0.0004% to 0.12% of the polymer, and the balance of the solvent.

A structural formula of the ionic liquid is shown in a formula (I):

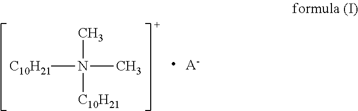

where A represents an organic or inorganic counter ion, and includes HCOO⁻, CH₃COO⁻, CH₃CH₂COO⁻, CH₃CH₂CH₂COO⁻, Cl⁻, Br⁻, or NO₃⁻.

The polymer includes at least one of polyethyleneimine, polyquaternium-7 (dimethyl diallyl ammonium chloride-acrylamide copolymer), polyarginine, or polylysine. The polyethyleneimine is BPEI or LPEI, and has an average molecular weight of 3,000 to 100,000. The polyarginine includes 6, 7, 9, or 11 polymerization units, or has a molecular weight of less than 2,000. The solvent includes water, an alcohol, or an ether. The water includes ultrapure water or DEPC-treated water. The alcohol includes 1,2-pentanediol, 1,3-propanediol, 1,2-butanediol, ethylene glycol, glycerin, or 1,2-hexanediol. The ether is isosorbide dimethyl ether.

Further, the composition of the present disclosure also includes a lipid and/or a polypeptide. In weight percentages, the composition further includes 0.011% to 0.33% of the lipid and/or 0.001% to 0.22% of the polypeptide. The lipid includes at least one of hydrogenated lecithin, soy lecithin, phosphatidylethanolamine, dipalmitoylphosphatidylcholine, distearoylphosphatidylcholine, or cholesterol. The polypeptide includes at least one of acetyl tetrapeptide-3, acetyl dipeptide-1 cetyl ester, biotinoyl tripeptide-1, or oligopeptide-54.

In the present disclosure, components in compositions 1 to 37 and weight percentages thereof are shown in Tables 3 below (contents in the table all refer to mass percentage contents), components in compositions 1 to 13 and weight percentages thereof are shown in Table 3-1 below, components in compositions 14 to 24 and weight percentages thereof are shown in Table 3-2 below, components in compositions 25 to 28 and weight percentages thereof are shown in Table 3-3 below, and components in compositions 29 to 37 and weight percentages thereof are shown in Table 3-4 below.

TABLE 3-1

| | | Composition | Composition | Composition | Composition | Composition | Composition | Composition |
|---|---|---|---|---|---|---|---|---|
| | | 1 | 2 | 3 | 4 | 5 | 6 | 7 |
| Nucleic | Substance | si-AR-3 | si-AR-3 | PC-AR-1 | si-AR-2 | si-AR-1 | PC-AR-1 | PC-AR-2 |
| acid | Content | 0.0008 | 0.00008 | 0.012 | 0.0008 | 0.0008 | 0.0008 | 0.0008 |
| Ionic | Substance | Didecyldi- | Didecyldi- | Didecyldi- | Didecyldi- | Didecyldi- | Didecyldi- | Didecyldi- |
| liquid | | methyl- | methyl- | methyl- | methyl- | methyl- | methyl- | methyl- |
| | | ammonium | ammonium | ammonium | ammonium | ammonium | ammonium | ammonium |
| | | chloride | formate | nitrate | bromide | formate | nitrate | chloride |
| | Content | 0.048 | 0.0002 | 5 | 0.004 | 0.004 | 0.004 | 0.008 |
| Polymer | Substance | BPEI | Polyquaternium-7 | Polyarginine-9 | LPEI | BPEI | Polylysine | LPEI |
| | | (10,000) | | | (10,000) | (30,000 to | | (30,000 to |
| | | | | | | 70,000) | | 70,000) |
| | Content | 0.036 | 0.0004 | 0.12 | 0.009 | 0.005 | 0.005 | 0.05 |
| Solvent | Ultrapure | To100 | — | To100 | — | — | — | — |
| | water | | | | | | | |
| | DEPC- | — | To100 | — | To100 | To100 | To100 | To100 |
| | treated | | | | | | | |
| | water | | | | | | | |
| | 1,2- | 5 | — | 5 | 5 | 5 | 5 | 5 |
| | Pentanediol | | | | | | | |
| | 1,3- | — | 30 | — | — | — | — | — |
| | Propanediol | | | | | | | |
| | 1,2- | — | — | — | — | — | 15 | — |
| | Butanediol | | | | | | | |
| | Ethylene | — | — | — | — | 15 | — | — |
| | glycol | | | | | | | |
| | Isosorbidedi- | — | — | 3 | — | — | — | 3 |
| | methyl | | | | | | | |
| | ether | | | | | | | |
| | Glycerin | 30 | — | 15 | 15 | — | — | 15 |
| | 1,2- | — | 0.5 | — | — | — | — | — |
| | Hexanediol | | | | | | | |

| | | Composition | Composition | Composition | Composition | Composition | Composition |
|---|---|---|---|---|---|---|---|
| | | 8 | 9 | 10 | 11 | 12 | 13 |
| Nucleic | Substance | PC-AR-3 | asi-AR-1 | asi-AR-2 | asi-AR-3 | si-AR-3 | PC-AR-1 |
| acid | Content | 0.0008 | 0.0008 | 0.0008 | 0.0008 | 0.0008 | 0.0008 |
| Ionic | Substance | Didecyldi- | Didecyldi- | Didecyldi- | Didecyldi- | Didecyldi- | Didecyldi- |
| liquid | | methyl- | methyl- | methyl- | methyl- | methyl- | methyl- |
| | | ammonium | ammonium | ammonium | ammonium | ammonium | ammonium |
| | | chloride | bromide | bromide | chloride | chloride | chloride |
| | Content | 0.12 | 0.004 | 0.004 | 0.004 | 0.004 | 0.004 |
| Polymer | Substance | BPEI | LPEI | Polyarginine-6 | BPEI | Polyarginine-7 | Polyarginine-11 |
| | | (100,000) | (100,000) | | (3,000) | | |
| | Content | 0.12 | 0.05 | 0.05 | 0.048 | 0.05 | 0.05 |
| Solvent | Ultrapure | — | — | — | — | To100 | To100 |
| | water | | | | | | |
| | DEPC- | To100 | To100 | To100 | To100 | — | — |
| | treated | | | | | | |
| | water | | | | | | |
| | 1,2- | 5 | 5 | 5 | 5 | 5 | 5 |
| | Pentanediol | | | | | | |
| | 1,3- | — | — | — | — | — | — |
| | Propanediol | | | | | | |
| | 1,2- | — | — | — | — | — | — |
| | Butanediol | | | | | | |
| | Ethylene | — | — | — | — | — | — |
| | glycol | | | | | | |
| | Isosorbidedi- | — | 2 | — | 5 | — | — |
| | methyl | | | | | | |
| | ether | | | | | | |
| | Glycerin | 15 | 15 | 15 | 15 | 30 | 30 |
| | 1,2- | 0.5 | 1 | — | 0.5 | — | — |
| | Hexanediol | | | | | | |

TABLE 3-2

| | | Composition | Composition | Composition | Composition | Composition | Composition |
|---|---|---|---|---|---|---|---|
| | | 14 | 15 | 16 | 17 | 18 | 19 |
| Nucleic | Substance | si-AR-3 | si-AR-3 | PC-AR-1 | si-AR-3 | PC-AR-1 | si-AR-3 |
| acid | Content | 0.0008 | 0.0008 | 0.0008 | 0.0008 | 0.0008 | 0.0008 |
| Lipid | Hydrogenated | 0.05 | 0.01 | 0.1 | — | — | — |
| | lecithin | | | | | | |
| | Soy | — | — | — | PC90 | PC90 | — |
| | lecithin | | | | | | |
| | Content | — | — | — | 0.02 | 0.3 | — |
| | Phosphatidyl- | — | — | — | — | — | 0.01 |
| | ethanolamine | | | | | | |
| | Dipalmitoyl- | — | — | — | — | — | — |
| | phosphatidyl- | | | | | | |
| | choline | | | | | | |
| | Distearoyl- | — | — | — | — | — | — |
| | phosphatidyl- | | | | | | |
| | choline | | | | | | |
| | Cholesterol | 0.01 | 0.001 | 0.01 | 0.002 | 0.03 | 0.005 |
| Ionic | Substance | Didecyldi- | Didecyldi- | Didecyldi- | Didecyldi- | Didecyldi- | Didecyldi- |
| liquid | | methyl- | methyl- | methyl- | methyl- | methyl- | methyl- |
| | | ammonium | ammonium | ammonium | ammonium | ammonium | ammonium |
| | | chloride | bromide | chloride | formate | nitrate | chloride |
| | Content | 0.048 | 0.004 | 0.004 | 0.004 | 0.004 | 0.008 |
| Polymer | Substance | BPEI | BPEI | Polyquaternium-7 | Polyarginine-9 | Polylysine | Polylysine |
| | | (10,000) | (10,000) | | | | |
| | Content | 0.036 | 0.008 | 0.009 | 0.005 | 0.005 | 0.05 |
| Solvent | Ultrapure | To 100 | — | — | — | — | — |
| | water | | | | | | |
| | DEPC- | — | To 100 | To 100 | To 100 | To 100 | To 100 |
| | treated | | | | | | |
| | water | | | | | | |
| | 1,2- | 5 | 5 | 5 | 5 | 5 | 5 |
| | Pentanediol | | | | | | |
| | 1,3- | — | 15 | — | — | — | — |
| | Propanediol | | | | | | |
| | 1,2- | — | — | — | — | 15 | — |
| | Butanediol | | | | | | |
| | Ethylene | — | — | — | 15 | — | — |
| | glycol | | | | | | |
| | Isosorbide | — | — | — | — | — | 3 |
| | dimethyl | | | | | | |
| | ether | | | | | | |
| | Glycerin | 30 | — | 15 | — | — | 15 |
| | 1,2- | — | — | — | — | — | — |
| | Hexanediol | | | | | | |

| | | Composition | Composition | Composition | Composition | Composition |
|---|---|---|---|---|---|---|
| | | 20 | 21 | 22 | 23 | 24 |
| Nucleic | Substance | PC-AR-1 | si-AR-3 | PC-AR-1 | si-AR-3 | PC-AR-1 |
| acid | Content | 0.0008 | 0.0008 | 0.0008 | 0.0008 | 0.00008 |
| Lipid | Hydrogenated | — | — | — | — | — |
| | lecithin | | | | | |
| | Soy | PC50 | — | PC70 | — | — |
| | lecithin | | | | | |
| | Content | 0.1 | — | 0.1 | — | — |
| | Phosphatidyl- | — | — | — | — | — |
| | ethanolamine | | | | | |
| | Dipalmitoyl- | — | 0.01 | — | — | — |
| | phosphatidyl- | | | | | |
| | choline | | | | | |
| | Distearoyl- | — | — | — | 0.01 | 0.1 |
| | phosphatidyl- | | | | | |
| | choline | | | | | |
| | Cholesterol | 0.02 | 0.002 | 0.02 | 0.03 | 0.01 |
| Ionic | Substance | Didecyldi- | Didecyldi- | Didecyldi- | Didecyldi- | Didecyldi- |
| liquid | | methyl- | methyl- | methyl- | methyl- | methyl- |
| | | ammonium | ammonium | ammonium | ammonium | ammonium |
| | | chloride | bromide | bromide | chloride | chloride |
| | Content | 0.12 | 0.004 | 0.004 | 0.004 | 0.0002 |
| Polymer | Substance | Polyquaternium-7 | Polyarginine-9 | BPEI | Polyquaternium-7 | Polyarginine-9 |
| | | | | (10,000) | | |
| | Content | 0.12 | 0.05 | 0.008 | 0.048 | 0.0005 |
| Solvent | Ultrapure | — | — | — | — | — |
| | water | | | | | |
| | DEPC- | To 100 | To 100 | To 100 | To 100 | To 100 |
| | treated | | | | | |
| | water | | | | | |
| | 1,2- | 5 | 5 | 5 | 5 | — |
| | Pentanediol | | | | | |
| | 1,3- | — | — | — | — | 30 |
| | Propanediol | | | | | |
| | 1,2- | — | — | — | — | — |
| | Butanediol | | | | | |
| | Ethylene | — | — | — | — | — |
| | glycol | | | | | |
| | Isosorbide | — | 2 | — | 5 | — |
| | dimethyl | | | | | |
| | ether | | | | | |
| | Glycerin | 15 | 15 | 15 | 15 | — |
| | 1,2- | 0.5 | 1 | — | 0.5 | 0.5 |
| | Hexanediol | | | | | |

TABLE 3-3

| | | Composition 25 | Composition 26 | Composition 27 | Composition 28 |
|---|---|---|---|---|---|
| Nucleic | Substance | si-AR-3 | PC-AR-1 | si-AR-3 | PC-AR-1 |
| acid | Content | 0.0008 | 0.00008 | 0.0008 | 0.00008 |
| Ionic | Substance | Didecyldimethylammonium | Didecyldimethylammonium | Didecyldimethylammonium | Didecyldimethylammonium |
| liquid | | chloride | nitrate | formate | bromide |
| | Content | 0.3 | 0.3 | 0.3 | 0.3 |
| Polymer | BPEI (with a | 0.08 | — | — | 0.0004 |
| | molecular | | | | |
| | weight of | | | | |
| | 10,000) | | | | |
| | Polyquaternium-7 | — | 0.0009 | 0.0012 | — |
| Solvent | Ultrapure water | To 100 | To 100 | To 100 | To 100 |
| | DEPC-treated | — | — | — | — |
| | water | | | | |
| | 1,2-Pentanediol | — | 5 | — | — |
| | 1,3-Propanediol | 15 | — | — | — |
| | 1,2-Butanediol | — | — | 30 | — |
| | Ethylene glycol | — | — | — | 30 |
| | Isosorbide | 10 | — | — | — |
| | dimethyl ether | | | | |
| | Glycerin | — | 30 | — | — |
| | 1,2-Hexanediol | 0.5 | 0.5 | 1 | 1 |
| Polypeptide | Acetyl | 0.001 | 0.2 | | 0.02 |
| | tetrapeptide-3 | | | | |
| | Acetyl dipeptide-1 | — | — | 0.001 | — |
| | cetyl ester | | | | |
| | Biotinoyl | — | — | 0.001 | 0.2 |
| | tripeptide-1 | | | | |
| | Oligopeptide-54 | — | — | 0.01 | — |

TABLE 3-4

| | | Composition | Composition | Composition | Composition | Composition |
|---|---|---|---|---|---|---|
| | | 29 | 30 | 31 | 32 | 33 |
| Nucleic | Substance | si-AR-3 | PC-AR-1 | si-AR-3 | PC-AR-1 | si-AR-3 |
| acid | Content | 0.0008 | 0.0008 | 0.0008 | 0.00008 | 0.0008 |
| Lipid | Hydrogenated | 0.05 | 0.02 | 0.02 | 0.02 | 0.02 |
| | lecithin | | | | | |
| | Distearoyl- | — | — | 0.01 | 0.1 | — |
| | phosphatidyl- | | | | | |
| | choline | | | | | |
| | Cholesterol | 0.01 | 0.002 | 0.002 | 0.002 | 0.002 |
| Ionic | Substance | Didecyldi- | Didecyldi- | Didecyldi- | Didecyldi- | Didecyldi- |
| liquid | | methyl- | methyl- | methyl- | methyl- | methyl- |
| | | ammonium | ammonium | ammonium | ammonium | ammonium |
| | | chloride | bromide | chloride | chloride | chloride |
| | Content | 0.048 | 0.004 | 0.004 | 0.0002 | 0.048 |
| Polymer | Substance | BPEI | BPEI | Polyquaternium-7 | Polyarginine-9 | BPEI |
| | | (10,000) | (10,000) | | | (10,000) |
| | Content | 0.036 | 0.008 | 0.048 | 0.0005 | 0.036 |
| Solvent | Ultrapure | To 100 | To 100 | To 100 | To 100 | To 100 |
| | water | | | | | |
| | 1,2- | 5 | 5 | 5 | — | 5 |
| | Pentanediol | | | | | |
| | 1,3- | — | — | — | 30 | — |
| | Propanediol | | | | | |
| | 1,2- | — | — | — | — | — |
| | Butanediol | | | | | |
| | Ethylene | — | — | — | — | — |
| | glycol | | | | | |
| | Isosorbide | — | — | 5 | — | — |
| | dimethyl | | | | | |
| | ether | | | | | |
| | Glycerin | 30 | 15 | 15 | — | 30 |
| | 1,2- | — | — | 0.5 | 0.5 | — |
| | Hexanediol | | | | | |
| Polypeptide | Acetyltetra- | 0.2 | 0.001 | 0.2 | — | 0.02 |
| | peptide-3 | | | | | |
| | Acetyldi- | — | — | — | 0.001 | — |
| | peptide-1 | | | | | |
| | cetyl ester | | | | | |
| | Biotinoyltri- | — | — | — | 0.001 | 0.2 |
| | peptide-1 | | | | | |
| | Oligopep- | — | — | — | 0.01 | — |
| | tide-54 | | | | | |

| | | Composition | Composition | Composition | Composition |
|---|---|---|---|---|---|
| | | 34 | 35 | 36 | 37 |
| Nucleic | Substance | PC-AR-1 | si-AR-3 | PC-AR-1 | si-AR-3 |
| acid | Content | 0.012 | 0.0008 | 0.0004 | 0.004 |
| Lipid | Hydrogenated | 0.02 | 0.02 | 0.02 | 0.02 |
| | lecithin | | | | |
| | Distearoyl- | — | — | — | — |
| | phosphatidyl- | | | | |
| | choline | | | | |
| | Cholesterol | 0.002 | 0.002 | 0.002 | 0.002 |
| Ionic | Substance | Didecyldi- | Didecyldi- | Didecyldi- | Didecyldi- |
| liquid | | methyl- | methyl- | methyl- | methyl- |
| | | ammonium | ammonium | ammonium | ammonium |
| | | chloride | chloride | formate | bromide |
| | Content | 0.3 | 0.05 | 0.3 | 0.3 |
| Polymer | Substance | BPEI | Polyquaternium-7 | Polyquaternium-7 | BPEI |
| | | (10,000) | | | (10,000) |
| | Content | 0.08 | 0.0009 | 0.0012 | 0.0004 |
| Solvent | Ultrapure | To 100 | To 100 | To 100 | To 100 |
| | water | | | | |
| | 1,2- | — | 5 | — | — |
| | Pentanediol | | | | |
| | 1,3- | 15 | — | — | — |
| | Propanediol | | | | |
| | 1,2- | — | — | 30 | — |
| | Butanediol | | | | |
| | Ethylene | — | — | — | 30 |
| | glycol | | | | |
| | Isosorbide | 10 | — | — | — |
| | dimethyl | | | | |
| | ether | | | | |
| | Glycerin | — | 30 | — | — |
| | 1,2- | 0.5 | 0.5 | — | — |
| | Hexanediol | | | | |
| Polypeptide | Acetyltetra- | — | — | 0.2 | — |
| | peptide-3 | | | | |
| | Acetyldi- | — | 0.1 | — | — |
| | peptide-1 | | | | |
| | cetyl ester | | | | |
| | Biotinoyltri- | — | 0.05 | — | 0.01 |
| | peptide-1 | | | | |
| | Oligopep- | 0.2 | — | — | 0.001 |
| | tide-54 | | | | |

A preparation method for the compositions 1 to 10 was provided, including the following steps:
S1: A polymer and a nucleic acid were dissolved with a part of a solvent separately to produce a polymer solution and a nucleic acid solution. The polymer solution and the nucleic acid solution were refrigerated at 2° C. to 8° C.
S2: The ionic liquid was mixed with a remaining part of the solvent, and heating was conducted at 40° C. to 60° C. for complete dissolution to produce a mixture. The mixture was refrigerated at 2° C. to 8° C.
S3: A refrigerated polymer solution obtained in the S1 was added to a refrigerated mixture obtained in the S2, and thorough mixing was allowed. A refrigerated nucleic acid solution obtained in the S1 was added, and incubation was allowed for 1 h to 4 h to produce a composition. The composition was stored in a −15° C. environment.

A preparation method for the compositions 11 to 13 was provided, including the following steps:
S1: A polymer and a nucleic acid were dissolved with a part of a solvent separately to produce a polymer solution and a nucleic acid solution. The polymer solution and the nucleic acid solution were refrigerated at 0° C. to 10° C.
S2: The ionic liquid was mixed with a remaining part of the solvent, and complete dissolution was allowed to produce a mixture. The mixture was refrigerated at 0° C. to 10° C.
S3: A refrigerated polymer solution obtained in the S1 was added to a refrigerated mixture obtained in the S2, and thorough mixing was allowed. A refrigerated nucleic acid solution obtained in the S1 was added, and incubation was allowed for 1 h to 4 h to produce a composition. The composition was stored in a −15° C. environment.

A preparation method for the compositions 14 to 24 was provided, including the following steps:
S1: A polymer and a nucleic acid were dissolved with a part of a solvent separately to produce a polymer solution and a nucleic acid solution. The polymer solution and the nucleic acid solution were refrigerated at 2° C. to 8° C.
S2: A lipid, an ionic liquid, and a part of the solvent were mixed, and complete dissolution was allowed at 40° C. to 60° C. to produce a mixture. A remaining part of the solvent at 40° C. to 60° C. was added to the mixture, and stirring was conducted for 5 min to 10 min. High-speed shearing was conducted at 3,000 r/min to 5,000 r/min for 1 min to 3 min, and high-pressure homogenization was conducted 2 times to 6 times at 200 bar to 600 bar to produce a homogeneous mixture. The homogeneous mixture was refrigerated at 2° C. to 8° C.
S3: A refrigerated polymer solution obtained in the S1 was added to a refrigerated homogeneous mixture obtained in the S2, and thorough mixing was allowed. A refrigerated nucleic acid solution obtained in the S1 was added, and incubation was allowed for 1 h to 4 h to produce a composition. The composition was stored in a −15° C. environment.

A preparation method for the compositions 25 to 28 was provided, including the following steps:
S1: A polymer, a nucleic acid, and a polypeptide were dissolved with a part of a solvent separately to produce a polymer solution, a nucleic acid solution, and a polypeptide solution. The polymer solution, the nucleic acid solution, and the polypeptide solution were refrigerated at 2° C. to 8° C.
S2: An ionic liquid was mixed with a remaining part of the solvent, and complete dissolution was allowed at 40° C. to 60° C. to produce a mixture. The mixture was refrigerated at 2° C. to 8° C.
S3: A refrigerated polymer solution obtained in the S1 was added to a refrigerated mixture obtained in the S2, and thorough mixing was conducted. A refrigerated polypeptide solution obtained in the S1 was added, and thorough mixing was conducted. A refrigerated nucleic acid solution obtained in the S1 was added, and incubation was allowed for 1 h to 4 h to produce a composition. The composition was stored in a −15° C. environment.

A preparation method for the compositions 29 to 37 was provided, including the following steps:
S1: A polymer, a nucleic acid, and a polypeptide were dissolved with a part of a solvent separately to produce a polymer solution, a nucleic acid solution, and a polypeptide solution. The polymer solution, the nucleic acid solution, and the polypeptide solution were refrigerated at 2° C. to 8° C.
S2: A lipid, an ionic liquid, and a part of the solvent were mixed, and complete dissolution was allowed at 40° C. to 60° C. to produce a mixture. A remaining part of the solvent at 40° C. to 60° C. was added to the mixture, and stirring was conducted for 5 min to 10 min. High-speed shearing was conducted at 3,000 r/min to 5,000 r/min for 1 min to 3 min, and high-pressure homogenization was conducted 2 times to 6 times at 200 bar to 600 bar to produce a homogeneous mixture. The homogeneous mixture was refrigerated at 2° C. to 8° C.
S3: A refrigerated polymer solution obtained in the S1 was added to a refrigerated homogeneous mixture obtained in the S2, and thorough mixing was conducted. A refrigerated polypeptide solution obtained in the S1 was added, and thorough mixing was conducted. A refrigerated nucleic acid solution obtained in the S1 was added, and incubation was allowed for 1 h to 4 h to produce a composition. The composition was stored in a −15° C. environment.

### Comparative Example 1

A composition in Comparative Example 1 was the same as the composition 1, except that the polymer was absent. A preparation method of the composition in Comparative Example 1 was the same as the preparation method of the composition 1, except that the polymer was not added.

### Comparative Example 2

A composition in Comparative Example 2 was the same as the composition 14, except that the ionic liquid was absent. A preparation method of the composition in Comparative Example 2 was the same as the preparation method of the composition 14, except that the ionic liquid was not added.

### Comparative Example 3

A composition in Comparative Example 3 was the same as the composition 14, except that a mass percentage of the hydrogenated lecithin in the lipid was adjusted from 0.05% to 0.4%. A preparation method of the composition in Comparative Example 3 was the same as the preparation method of the composition 14.

### Comparative Example 4

A composition in Comparative Example 4 was the same as the composition 14. However, the composition in Comparative Example 4 was prepared by a different method including the following steps:
1) A polymer and a nucleic acid were dissolved with a part of a solvent separately to produce a polymer solution and a nucleic acid solution. The polymer solution and the nucleic acid solution were refrigerated at 2° C. to 8° C.
2) A lipid raw material was weighed proportionally and fully dissolved with 20 mL of chloroform to produce a lipid solution.
3) The lipid solution was heated from room temperature to 50° C. and subjected to rotary evaporation for removing the chloroform.
4) A remaining part of the solvent was added, stirring was fully conducted, and an ultrasonic hydration treatment was conducted for 2 h.
5) At room temperature, a refrigerated polymer solution was added, and shaking was conducted for 2 min. A resulting mixed system was stored overnight at 4° C.
6) A refrigerated nucleic acid solution was finally added, and static incubation was allowed at room temperature for 2 h. Filtration was conducted twice with a 0.45 μm water-based nylon to produce the composition, which was stored at −15° C.

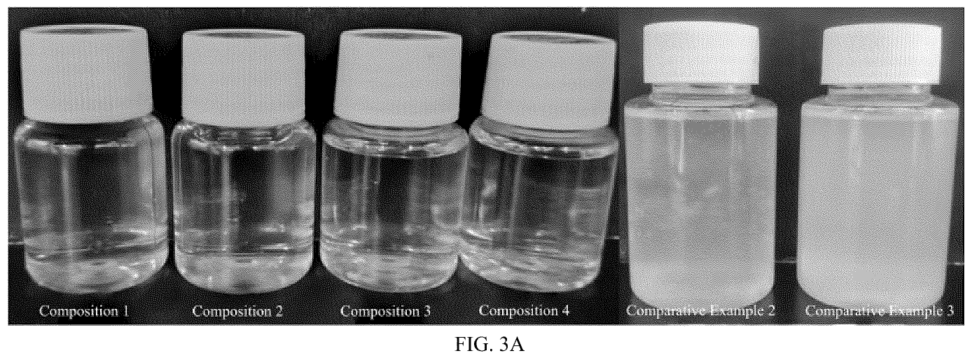

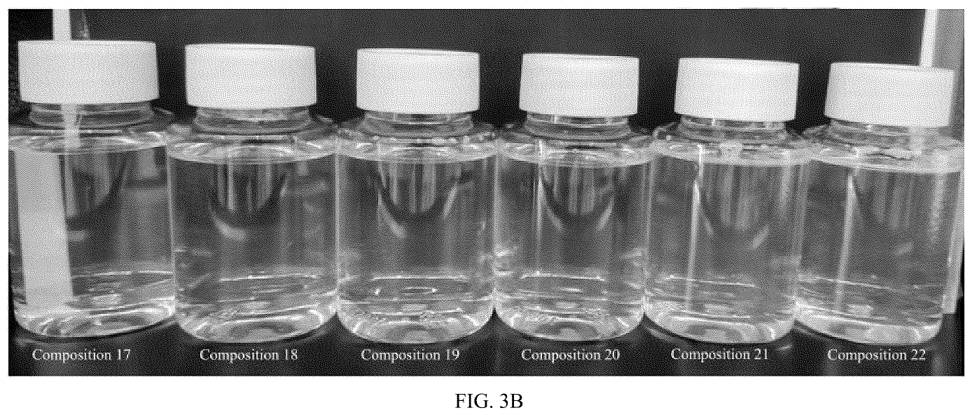

The compositions 1 to 37 prepared in Example 2 are translucent to transparent liquids. The composition in Comparative Example 1 is a transparent liquid. The compositions in Comparative Examples 2 and 3 are white opaque liquids with flocci. The composition in Comparative Example 4 is a translucent liquid. The appearance of the compositions 1 to 4 and 17 to 22 and the compositions in Comparative Examples 2 and 3 are shown in FIGS. 3A-3B. FIG. 3A shows the compositions 1 to 4 and the compositions in Comparative Examples 2 and 3 and FIG. 3B shows the compositions 17 to 22.

### Test Example 1

The encapsulation efficiencies (EEs), physical and chemical parameters, and stability of the compositions 1 to 37 in Example 2 and the compositions in Comparative Examples 1 to 4 were tested.

### 1. Particle Size, Zeta Potential, EE, pH Value, and Appearance Stability

A pH value was tested by a pH meter (Mettler Toledo, model: FE-38). A particle size and a polydispersity index (PDI) were tested by a laser particle size analyzer (Anton Paar, model: Litesizer 500). A zeta potential was tested by a zeta potential analyzer.

3′ FAM-labeled si-AR-3 was uniformly selected for EE testing (since siRNA and asiRNA were quite similar in terms of the molecular weight and hydrophilicity/hydrophobicity, siRNA with a relatively-large molecular weight was selected for testing). The EE was determined by the ultrafiltration centrifugation method with an ultrafiltration centrifuge tube of Millipore NMWL 10 KDa. Before the ultrafiltration centrifugation, a fluorescence intensity of a sample de-emulsified with 1% Triton X-100 was determined as A1. Ultrafiltration centrifugation was conducted for 5 min at 3,000 r/min and 0° C., and a filtrate was collected. A fluorescence intensity of the filtrate was determined as A2. The EE was calculated as follows: EE=(A1−A2)/A1 ×100%.

Appearance stability: Each composition was stored in a −15° C. freezer, and then thawed and restored to room temperature. Whether the appearance remained consistent with the initial state was observed weekly.

Test results for particle sizes, PDIs, zeta potentials, EEs, pH values, and appearance stability of the compositions 1 to 37 and the compositions in Comparative Examples 1 to 4 are shown in Tables 4 below. Test results for the compositions 1 to 13 and the composition in Comparative Example 1 are shown in Table 4-1 below. Test results for the compositions 14 to 24 and the compositions in Comparative Examples 2, 3, and 4 are shown in Table 4-2 below. Test results for the compositions 25 to 28 are shown in Table 4-3 below. Test results for the compositions 29 to 37 are shown in Table 4-4 below.

TABLE 4-1

| | Composition | Composition | Composition | Composition | Composition | Composition | Composition |
|---|---|---|---|---|---|---|---|
| Index | 1 | 2 | 3 | 4 | 5 | 6 | 7 |
| Particle | 294.1 | 280.8 | 368.9 | 335.8 | 345 | 373.4 | 349.1 |
| size, nm | | | | | | | |
| PDI, % | 25.7 (bimodal) | 19.4 (unimodal) | 25.5 (bimodal) | 13.8 (unimodal) | 25.1 (bimodal) | 23.0 (unimodal) | 25.9 (bimodal) |
| Zeta, mV | 13.8 | 12.2 | 18.4 | 17.4 | 12.5 | 19.8 | 14.6 |
| EE, % | 83.67 | 87.93 | 94.45 | 88.54 | 89.00 | 90.78 | 88.78 |
| pH | 6.02 | 6.45 | 6.77 | 6.04 | 6.34 | 7.56 | 6.25 |
| Appearance | Stable and | Stable and | Stable and | Stable and | Stable and | Stable and | Stable and |
| stability | transparent | transparent | transparent | transparent | transparent | transparent | translucent |

| | Composition | Composition | Composition | Composition | Composition | Composition | Comparative |
|---|---|---|---|---|---|---|---|
| Index | 8 | 9 | 10 | 11 | 12 | 13 | Example 1 |
| Particle | 285.2 | 296.5 | 357.4 | 350.3 | 429.4 | 389.9 | 231.4 |
| size, nm | | | | | | | |
| PDI, % | 27.4 (bimodal) | 26.4 (bimodal) | 24.0 (unimodal) | 22.6 (unimodal) | 26.3 (multimodal) | 28.9 (multimodal) | 22.4 (unimodal) |
| Zeta, mV | 15.0 | 16.5 | 16.9 | 14.3 | 14.3 | 15.1 | 11.5 |
| EE, % | 92.7 | 90.26 | 82.25 | 81.67 | 83.49 | 90.65 | 55.34 |
| pH | 6.38 | 6.23 | 6.30 | 6.32 | 6.41 | 6.86 | 6.35 |
| Appearance | Stable and | Stable and | Stable and | Stable and | Stable and | Stable and | Stable and |
| stability | translucent | translucent | translucent | transparent | transparent | transparent | transparent |

TABLE 4-2

| | Composition | Composition | Composition | Composition | Composition | Composition | Composition |
|---|---|---|---|---|---|---|---|
| Index | 14 | 15 | 16 | 17 | 18 | 19 | 20 |
| Particle | 205.0 | 211.2 | 239.8 | 195 | 205 | 243.7 | 180 |
| size, nm | | | | | | | |
| PDI, % | 22.2 (unimodal) | 20.5 (unimodal) | 21.7 (unimodal) | 20.6 (unimodal) | 22.2 (unimodal) | 10.4 (unimodal) | 7.3 (unimodal) |
| Zeta, mV | 13.6 | 14.9 | 15.8 | 13 | 19.8 | 12.9 | 14.2 |
| EE, % | 99.82 | 98.95 | 98.67 | 99.58 | 99.28 | 98.33 | 99.43 |
| pH | 6.56 | 6.50 | 6.82 | 6.30 | 6.13 | 6.17 | 6.42 |
| Appearance | Stable and | Stable and | Stable and | Stable and | Stable and | Stable and | Stable and |
| stability | transparent | transparent | transparent | transparent | transparent | transparent | transparent |

| | Composition | Composition | Composition | Composition | Comparative | Comparative | Comparative |
|---|---|---|---|---|---|---|---|
| Index | 21 | 22 | 23 | 24 | Example 2 | Example 3 | Example 4 |
| Particle | 294.6 | 343.2 | 276.9 | 303.6 | / | / | 384.27 |
| size, nm | | | | | | | |
| PDI, % | 23.6 (unimodal) | 20.7 (bimodal) | 23.1 (unimodal) | 18.9 (unimodal) | / | / | 27.9 (multimodal) |
| Zeta, mV | 16.6 | 16.5 | 13.7 | 14 | 2.6 | 15.3 | 14.3 |
| EE, % | 97.85 | 99.5 | 98.62 | 99.08 | / | / | 84.5 |
| pH | 6.55 | 6.74 | 6.38 | 6.07 | 6.78 | 6.27 | 6.20 |
| Appearance | Stable and | Stable and | Stable and | Stable and | It is unstable | It is unstable, | Stable and |
| stability | translucent | translucent | translucent | translucent | and there is | there is a | translucent |
| | | | | | a flocculent | stratification | |
| | | | | | precipitate | phenomenon, and | |
| | | | | | | there are flocci | |

TABLE 4-3

| Index | Composition 25 | Composition 26 | Composition 27 | Composition 28 |
|---|---|---|---|---|
| Particle size, nm | 325.2 | 314.0 | 392.3 | 325.9 |
| PDI, % | 22.6 (bimodal) | 20.4 (bimodal) | 27.8 (bimodal) | 22.4 (bimodal) |
| Zeta, mV | 13.6 | 20.6 | 24.1 | 16.5 |
| EE, % | 88.57 | 82.53 | 85.72 | 83.86 |
| pH | 6.56 | 6.66 | 6.19 | 5.84 |
| Appearance | Stable and | Stable and | Stable and | Stable and |
| stability | transparent | transparent | transparent | transparent |

TABLE 4-4

| Index | Composition 29 | Composition 30 | Composition 31 | Composition 32 | Composition 33 |
|---|---|---|---|---|---|
| Particle size, nm | 230.6 | 205.6 | 234.6 | 182.3 | 235.0 |
| PDI, % | 20.1 (unimodal) | 23.4 (unimodal) | 17.8 (unimodal) | 8.5 (unimodal) | 11.6 (unimodal) |
| Zeta, mV | 13.1 | 17.8 | 13.2 | 24.9 | 15 |
| EE, % | 99.12 | 99.63 | 98.16 | 99.06 | 98.53 |
| pH | 5.98 | 6.54 | 6.43 | 6.70 | 6.43 |
| Appearance | Stable and | Stable and | Stable and | Stable and | Stable and |
| stability | transparent | transparent | transparent | transparent | transparent |

| Index | Composition 34 | Composition 35 | Composition 36 | Composition 37 | / |
|---|---|---|---|---|---|
| Particle size, nm | 228.3 | 233.5 | 179.5 | 188.3 | / |
| PDI, % | 9.1 (unimodal) | 8.2 (unimodal) | 10.2 (unimodal) | 14.1 (unimodal) | / |
| Zeta, mV | 14.7 | 35.1 | 12.3 | 17.6 | / |
| EE, % | 99.12 | 98.64 | 99.54 | 99.24 | / |
| pH | 6.23 | 6.87 | 5.97 | 5.89 | / |
| Appearance | Stable and | Stable and | Stable and | Stable and | / |
| stability | transparent | transparent | transparent | translucent | |

According to the results in Table 4-1, Table 4-2, Table 4-3, and Table 4-4, the compositions 1 to 37 have a particle size of 150 nm to 450 nm. A lipid-containing composition (compositions 14 to 24 and compositions 29 to 37) have a smaller particle size than a lipid-free composition (compositions 1 to 13 and compositions 25 to 28). The compositions 1 to 37 are mostly monodisperse in terms of particle sizes, and all have a positive zeta potential conducive to the tight binding of electronegative nucleic acids, and relatively high EE (higher than 80%). A composition including both the lipid and the ionic liquid (compositions 14 to 24 and compositions 29 to 37) has higher EE than a composition including the ionic liquid and no lipid (compositions 1 to 13 and compositions 25 to 28). A composition including both the ionic liquid and the polymer (compositions 1 to 13) have higher EE and a higher zeta potential than Comparative Example 1 including the ionic liquid and no polymer.

The presence of the lipid and the absence of the ionic liquid in a composition (Comparative Example 2) or a too-high content of the lipid (especially a phospholipid) in a composition (Comparative Example 3) will make a composition system instable.

Compared with the composition prepared by the traditional film evaporation method (Comparative Example 4), the lipid-containing composition prepared by the high-pressure homogenization method (composition 14) has a small and uniform particle size, relatively-high EE, a low zeta potential of positive charges, and low cytotoxicity, and can be prepared by a simple and time-saving process.

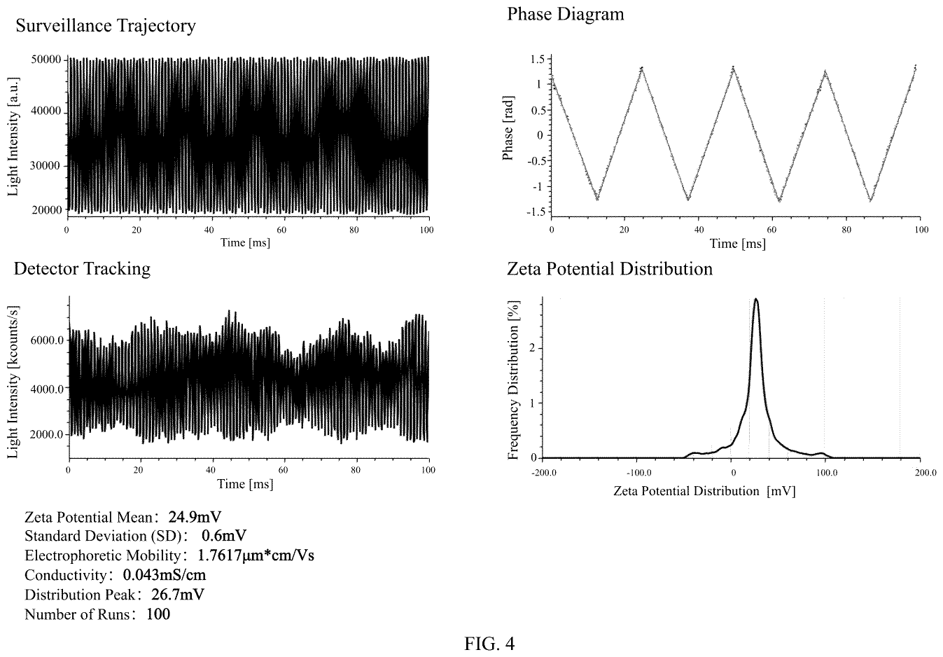

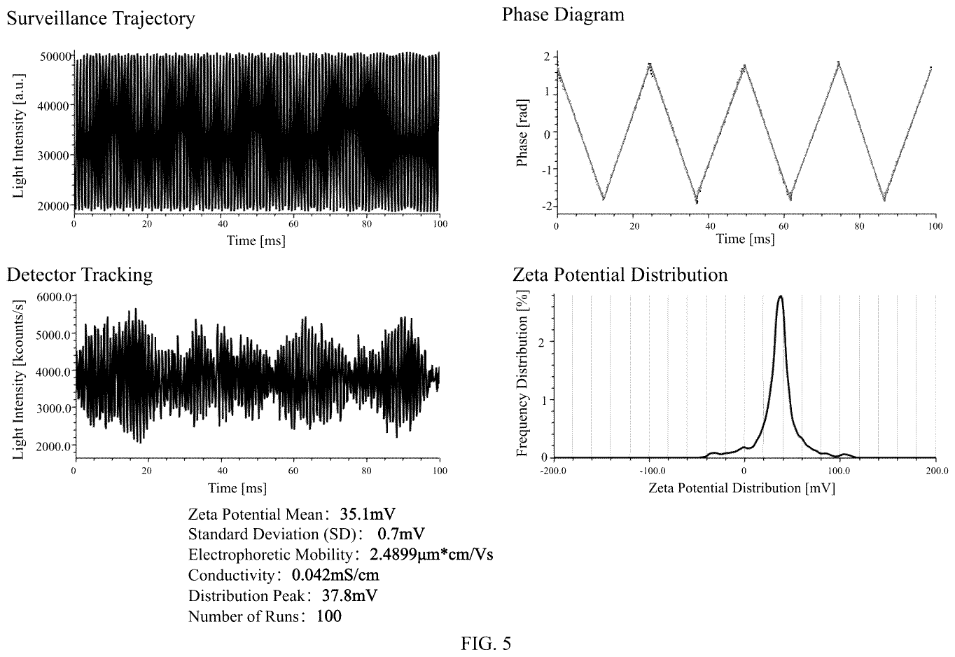

The compositions 27, 32, and 35 in Table 4-3 and Table 4-4 have relatively high zeta potentials, which is mainly attributed to the strong electropositivity of acetyl dipeptide-1 cetyl ester. Zeta potential measurement results of the composition 32 are shown in FIG. 4. Zeta potential measurement results of the composition 35 are shown in FIG. 5.

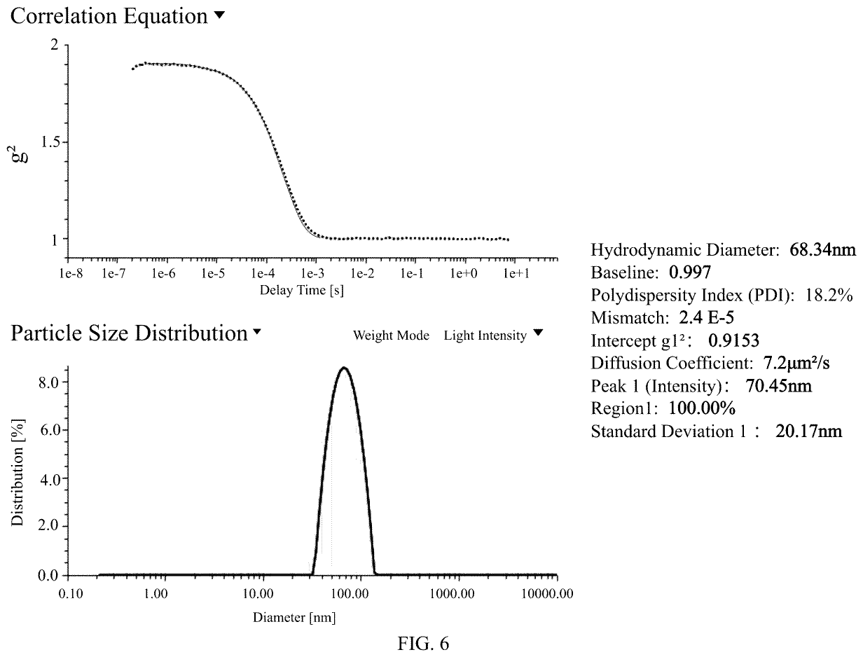

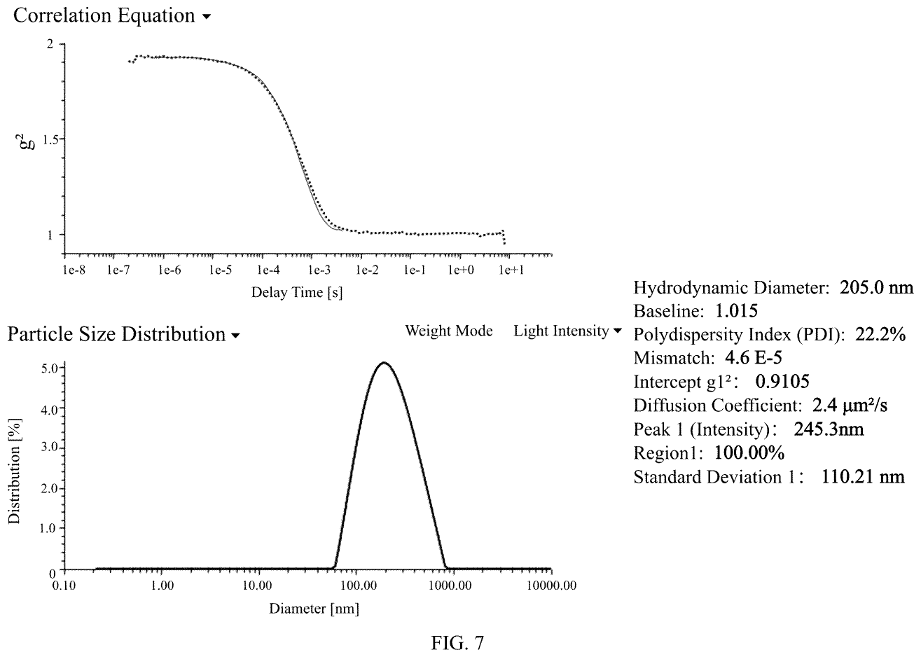

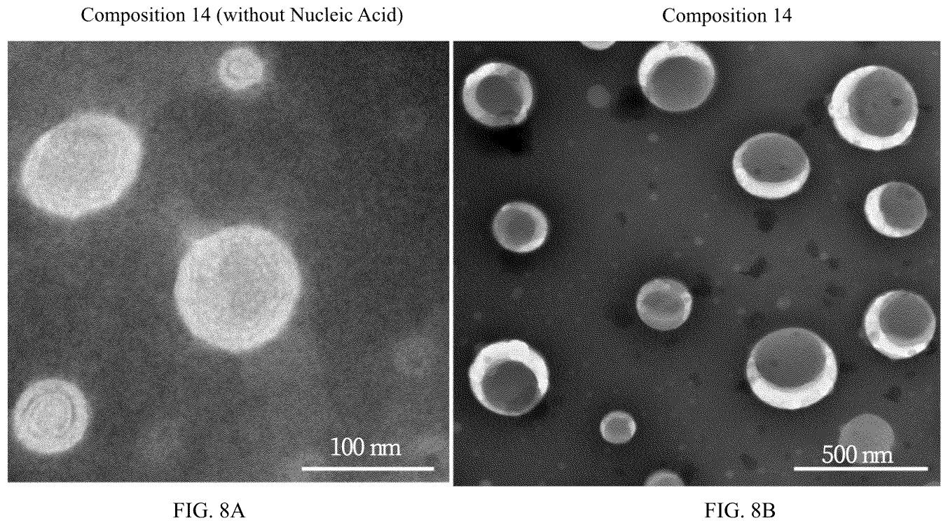

Particle sizes of the composition 14 before and after the addition of the nucleic acid were tested, and morphologies of the composition 14 before and after the addition of the nucleic acid were observed by TEM. According to test results, a mixture without the nucleic acid has a particle size of less than 100 nm, and a composition with the nucleic acid has a particle size of more than 150 nm. Particle size distribution results of the composition 14 before the addition of a nucleic acid are shown in FIG. 6. Particle size distribution results of the composition 14 after the addition of the nucleic acid are shown in FIG. 7. TEM images of the composition 14 before and after the addition of the nucleic acid are shown in FIGS. 8A-8B, where FIG. 8A shows the composition 14 without the nucleic acid and FIG. 8B shows the composition 14 with the nucleic acid. The results indicate that a nanostructure is formed in the composition 14.

### 2. Stability

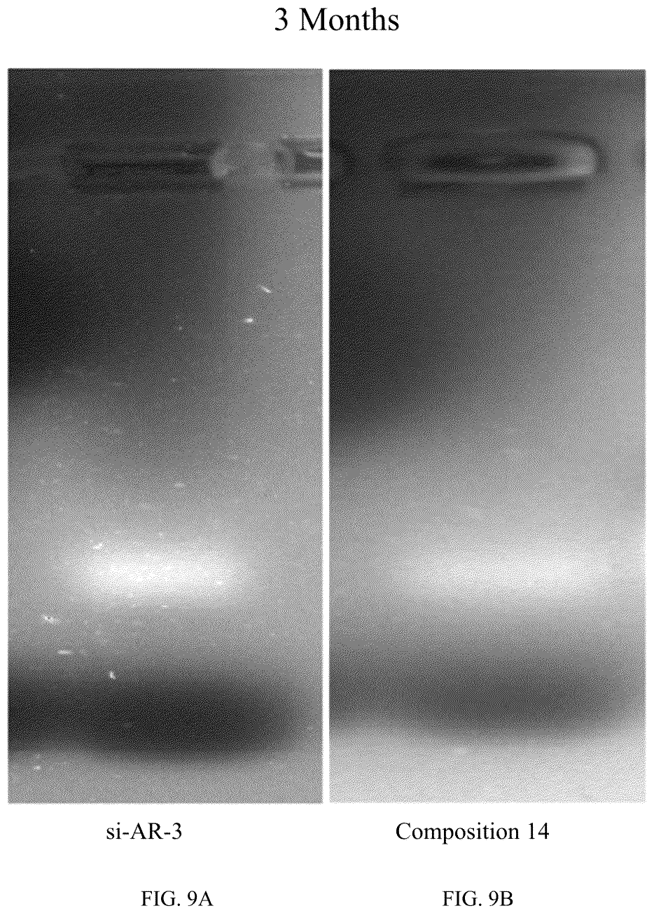

The compositions 1, 2, 14, 15, 29, and 35 were placed in a −15° C. environment for 3 months. A si-AR-3 nucleic acid powder was placed in a −25° C. environment for 3 months. All samples each were diluted to produce solutions with a same si-AR-3 concentration, and then subjected to a gel electrophoresis test. The compositions 1, 2, 14, 15, 29, and 35 placed in the −15° C. environment for 3 months had comparable gel electrophoresis results to the si-AR-3 nucleic acid powder placed in the −25° C. environment for 3 months, and si-AR-3 was not significantly degraded. Gel electrophoresis results of the composition 14 stored in the −15° C. environment for 3 months are shown in FIGS. 9A-9B, where FIG. 9A is for si-AR-4 and FIG. 9B is for the composition 14. It can be seen that the compositions 1 to 37 prepared by the present disclosure are stable when stored in the −15° C. environment.

### Test Example 2

The compositions 1, 7, 10, 14, 15, 29, and 35 in Example 2 were selected for patch testing. A human skin patch test was conducted in accordance with Safety and Technical Standards for Cosmetics, with ultrapure water as a control group. Patch testing results are shown in Table 5 below.

TABLE 5

| | | Skin reaction levels and | | | | |
|---|---|---|---|---|---|---|
| | Observation | corresponding numbers of people | | | | |
| Group | time | Score 0 | Score 1 | Score 2 | Score 3 | Score 4 |
| Composition 1 | 0.5 h | 33 | 0 | 0 | 0 | 0 |
| Composition 7 | | 33 | 0 | 0 | 0 | 0 |
| Composition 10 | | 33 | 0 | 0 | 0 | 0 |
| Composition 14 | | 33 | 0 | 0 | 0 | 0 |
| Composition 15 | | 33 | 0 | 0 | 0 | 0 |
| Composition 29 | | 33 | 0 | 0 | 0 | 0 |
| Composition 35 | | 33 | 0 | 0 | 0 | 0 |
| Control group | | 33 | 0 | 0 | 0 | 0 |

Notes:
Score 0: there is a negative reaction. Score 1: there is a doubtful reaction, including mild erythema only. Score 2: there is a weak positive reaction, including erythema, infiltration, edema, and possible papules. Score 3: there is a strong positive reaction, including erythema, infiltration, edema, and vesicles, and the reaction may exceed a test region. Score 4: there is an extremely strong positive reaction, including remarkable erythema, severe infiltration, edema, and coalescing vesicles, and the reaction exceeds a test region.

According to the results in Table 5, the compositions 1, 7, 10, 14, 15, 29, and 35 cause no adverse reactions to the human skin, indicating that the compositions of the present disclosure that do not include or include the lipid and/or include the polypeptide in addition to including the nucleic acid, the ionic liquid, the polymer, and the solvent all are safe for the human skin.

### Test Example 3

The skin permeation of the compositions 1 to 37 of Example 2 was tested.

The porcine dorsal ear skin was selected for testing (Franz diffusion cell transdermal absorption test). Nucleic acids adopted for all compositions in a fluorescent transdermal experiment are fluorescently labeled with FAM uniformly (FAM-nucleic acid). A mass proportion of each nucleic acid added was 0.008%. PBS was prepared with 0.1% DEPC-treated water, and 0.008% of FAM-nucleic acid was added to prepare a nucleic acid-containing PBS solution as a control. At 4 h and 16 h of incubation, a skin sample was collected, fixed overnight in 4% paraformaldehyde, washed with PBS, then quickly frozen at −80° C., vertically embedded with an optimal cutting temperature (OCT) compound, sectioned into full-thickness sections, and photographed by a laser scanning confocal microscope to observe the permeation of the sample in the skin.

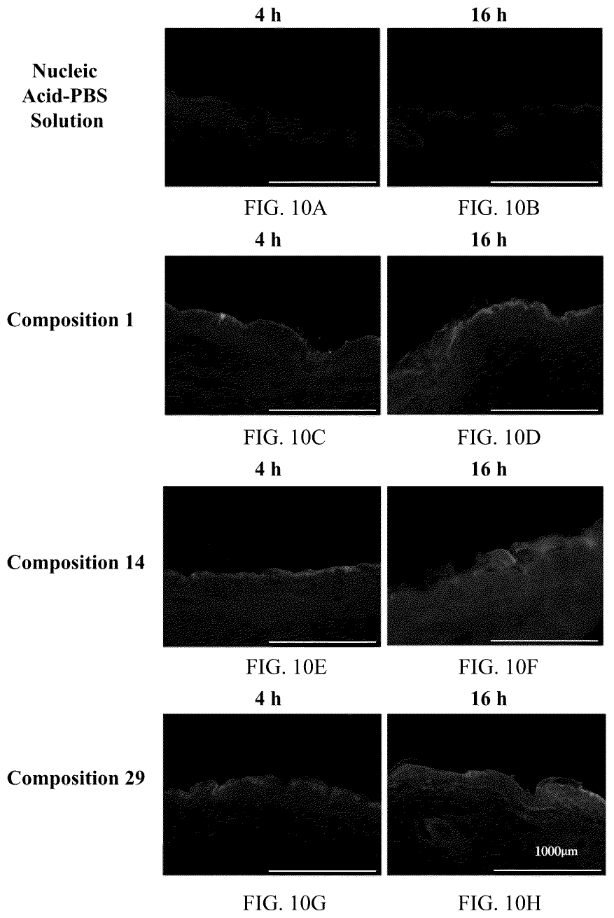

According to test results, compared with the ordinary PBS solution system, the compositions 1 to 37 can well promote the transdermal permeability of nucleic acids. The compositions 1 to 37 exhibit a 5 to 12 times higher fluorescence intensity than the PBS solution system. A penetration depth of a fluorescently-labeled nucleic acid increases from 50 μm to 150 μm (PBS solution control group) to 700 μm to 1,400 μm (compositions 1 to 37), indicating the significant enhancement in transdermal permeability. It can be known that a composition including the ionic liquid or the combination of the ionic liquid and the lipid has a prominent transdermal penetration effect, which is beneficial for the non-invasive transdermal drug delivery in vitro. Fluorescent transdermal results of the nucleic acid-containing PBS solution and the compositions 1, 14, and 29 at 4 h and 16 h are shown in FIGS. 10A-10H. FIG. 10A shows a fluorescent transdermal result of the nucleic acid-containing PBS solution at 4 h, FIG. 10B shows a fluorescent transdermal result of the nucleic acid-containing PBS solution at 16 h, FIG. 10C shows a fluorescent transdermal result of the composition 1 at 4 h, FIG. 10D shows a fluorescent transdermal result of the composition 1 at 16 h, FIG. 10E shows a fluorescent transdermal result of the composition 2 at 4 h, FIG. 10F shows a fluorescent transdermal result of the composition 2 at 16 h, FIG. 10G shows a fluorescent transdermal result of the composition 3 at 4 h, and FIG. 10H shows a fluorescent transdermal result of the composition 3 at 16 h.

### Test Example 4

### 1. Fluorescence Detection

According to the method in Example 1, the compositions 1 to 37 of Example 2 each were transfected into HFDPCs, and then the fluorescence was detected to observe whether a nucleic acid in a composition entered the HFDPCs. Each composition was labeled with FAM. A mass proportion of a nucleic acid added was 0.008%. A nucleic acid-containing PBS solution was adopted as a control (which was the same as Test Example 3).

The transfected cells were cultured in a 37° C. and 5% CO₂ incubator for 6 h, and then an Opti-MEM medium was discarded. Resulting cells were washed with PBS three times, fixed with 4% paraformaldehyde for 15 min, washed with PBS three times, stained with a cell membrane dye Did (5 μM) for 10 min, washed with PBS three times, stained with a cell nucleus dye DAPI (10 μg/mL) at room temperature in the dark for 5 min, then washed with PBS three times, and observed and photographed by a laser scanning confocal microscope (FAM-nucleic acid, green fluorescence, and excitation/emission wavelengths: 492 nm/518 nm).

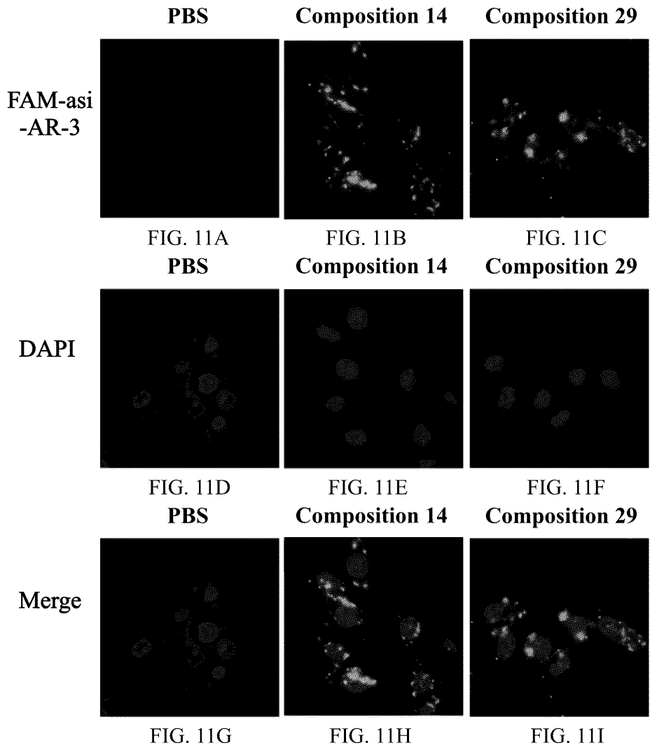

According to test results, cells transfected with the nucleic acid dissolved in the PBS solution do not exhibit fluorescence. However, according to fluorescence results of cells transfected with the compositions 1 to 37, the fluorescently-labeled nucleic acids partly enter the cell nuclei, and the cell nuclei remain relatively uniform circular shapes, indicating that the compositions have no obvious toxic and side effects for cells. Fluorescence results of cells transfected with the PBS and compositions 14 and 29 are shown in FIGS. 11A-11I. FIG. 11A shows a fluorescence result of FAM-asi-AR-3 for PBS, FIG. 11B shows a fluorescence result of FAM-asi-AR-3 for the composition 14, FIG. 11C shows a fluorescence result of FAM-asi-AR-3 for the composition 29, FIG. 11D shows a fluorescence result of DAPI for PBS, FIG. 11E shows a fluorescence result of DAPI for the composition 14, FIG. 11F shows a fluorescence result of DAPI for the composition 29, FIG. 11G shows a fluorescence result of Merge for PBS, FIG. 11H shows a fluorescence result of Merge for the composition 14, and FIG. 11I shows a fluorescence result of Merge for the composition 29.

### 2. Western Blot Assay

According to the method in Example 1, the compositions 1 to 37 of Example 2 each were transfected into HFDPCs. The Western blot assay was conducted to evaluate the silencing effects of the compositions 1 to 37 for the expression of the AR protein in normal HFDPCs.

According to test results, the compositions 1 to 37 can effectively silence the expression of the AR protein in normal HFDPCs, and the compositions including si-AR-2, si-AR-3, and PC-AR-1 exhibit a significant inhibitory effect on the AR protein.

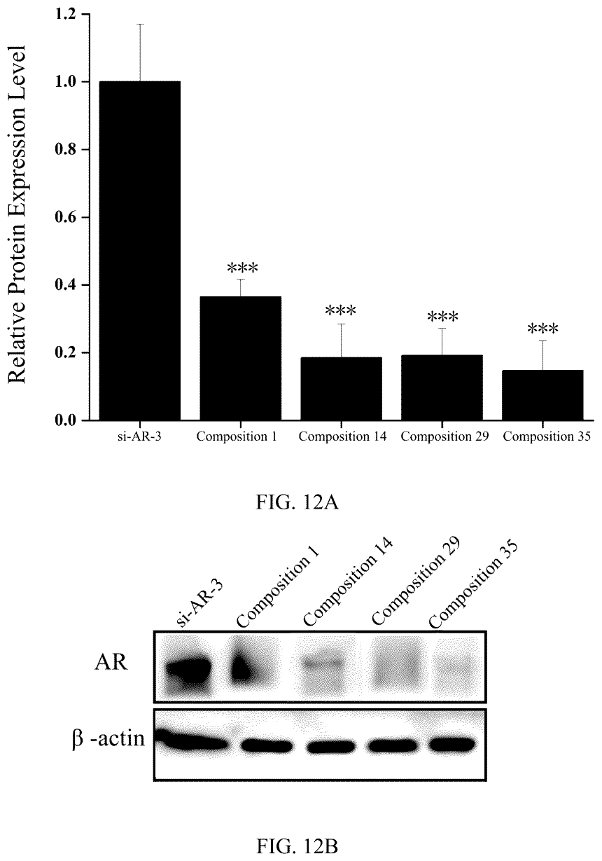

Relative expression levels of the AR protein in cells transfected with the compositions 1, 14, 29, and 35 are shown in FIGS. 12A-12B. FIG. 12A shows the quantitative analysis results, FIG. 12B shows the western blot results, and ***P<0.001.

### Test Example 5

Human HFDPCs were selected as the target cells for anti-hair loss efficacy. HFDPCs were stimulated with dihydrotestosterone (DHT, 100 nM) for 48 h to establish an AGA in vitro cell model (DHT model).

### 1. Influence of Compositions on the Expression of VEGF

The compositions 1 to 37 of Example 2 each were added to DHT model cells for synchronous investigation of the influence of the compositions on the expression of VEGF in the DHT model cells.

The compositions 1 to 37 with the same nucleic acid concentration (0.008%) each were added to the constructed DHT model cells. Resulting mixtures each were then incubated in a 37° C. and 5% CO₂ incubator for 8 h and then centrifuged. Resulting cell pellets were removed. Resulting culture supernatants were collected and tested for a VEGF concentration with a VEGF enzyme-linked immunosorbent assay (ELISA) kit.

According to test results, the expression of VEGF in HFDPCs significantly decreases after the stimulation with DTH. After the stimulation with DTH and the addition of the compositions 1 to 37, VEGF concentrations significantly increase compared to the model cells, with varying improvement levels. A composition including a nucleic acid with a prominent intrinsic silencing effect for the AR gene exhibits a pronounced promoting effect for the expression of VEGF. It can be seen that the addition of DHT inhibits the expression of normal VEGF in human HFDPCs, which compromises the nutrition supply to hair follicles. However, the addition of the composition of the present disclosure can significantly alleviate the negative impact that DHT reduces VEGF, thereby facilitating the nutrition supply to hair follicles and achieving the anti-hair loss effect.

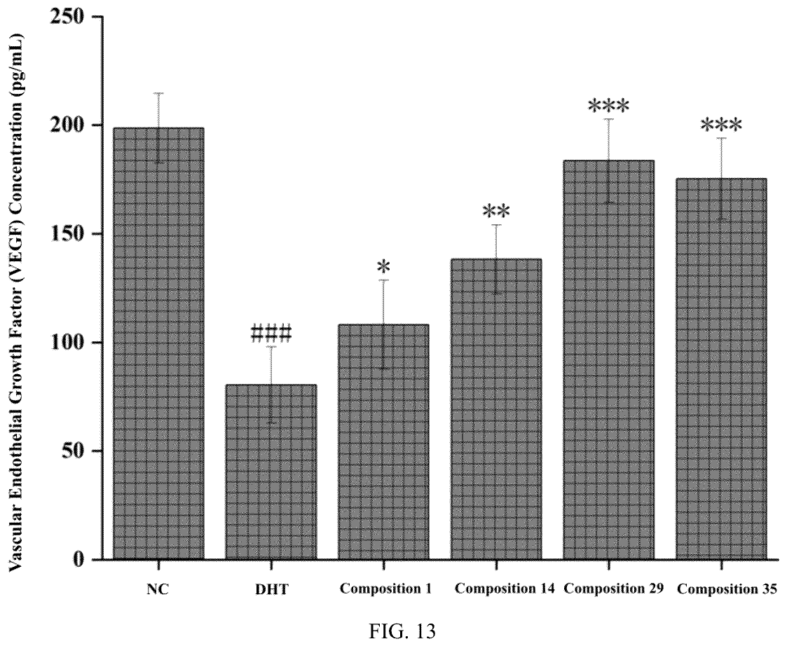

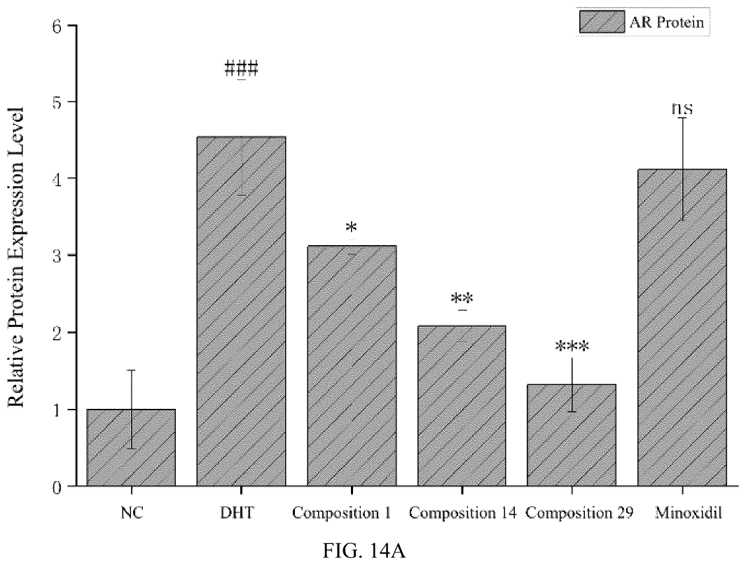

The expression levels of VEGF corresponding to the compositions 1, 14, 29, and 35 are shown in FIG. 13. In FIG. 13, NC represents normal HFDPCs, DHT represents DHT model cells, and compositions 1, 14, 29, and 35 represent DHT model cells treated with the compositions 1, 14, 29, and 35, respectively; P<0.05 is denoted as “*” or “#”; P<0.01 is denoted as “**” or “#”; and P<0.001 is denoted as “*” or “#” (where the significance between a model group and a control group is represented by “#” and the significance between different treatment groups or a positive control and the model group is represented by “*”, the same below).

### 2. Influence of Compositions on Hair Loss-Associated Proteins

The compositions 1 to 37 of Example 2 each were added to DHT model cells for synchronous investigation of the influence of the compositions on the target protein AR and the related proteins IL-1α, IL-8, TNF-α, DKK-1, TGF-β2, BMP4, β-catenin, and MMP3 in the DHT model cells.

The compositions 1 to 37 with the same nucleic acid concentration (0.008%) each were added to the constructed DHT model cells. Minoxidil was adopted as a positive control. Resulting mixtures each were incubated in a 37° C. and 5% CO₂ incubator for 8 h, and then centrifuged. Resulting cell pellets were collected, an RIPA lysis buffer was added, and lysis was allowed at 4° C. for 30 min. Centrifugation was conducted at 12,000 rpm for 10 min, and resulting supernatants were collected. The total cellular protein was extracted. The expression levels of the target protein and the related proteins were detected according to the protein detection method (Western blot) in Example 1.

According to test results, the expression of the target protein AR and the related proteins IL-1α, IL-8, and TNF-α significantly increases under DHT induction, but significantly decreases when the compositions 1 to 37 are added. The positive control minoxidil does not have a significant impact on AR. The expression of any of the related proteins DKK-1, TGF-β2, and BMP4 is significantly reduced. The composition 29 leads to a comparable or even better effect for reducing the expression of the related proteins to or than the positive control minoxidil. In addition, the expression of the related proteins β-catenin and MMP3 is significantly reduced under DHT induction, while the expression of these two proteins significantly increases after the addition of the compositions 1 to 37. The composition 14 or the composition 29 has a better effect than the positive control minoxidil. These results fully indicate that nucleic acids have a targeted silencing effect for the AR protein, and can inhibit or promote the expression of proteins related to hair loss, and these proteins are associated with multiple pathways including a β-catenin pathway, a BMP signaling pathway, a PI3K/AKT pathway, a TGF-β signaling pathway, and a Wnt signaling pathway, which achieves the comprehensive anti-hair loss effect. In contrast, minoxidil does not have an AR-targeting effect. According to test results, minoxidil affects the expression of DKK-1, TGF-β2, and BMP4 proteins. It can be known that the two anti-hair loss substances (nucleic acids and minoxidil) have different action mechanisms. Nucleic acids have more action pathways than minoxidil. Nucleic acids not only can directly silence the AR target genes, but also can further affect the expression of related factors downstream of the AR gene, creating a cascade amplification effect.

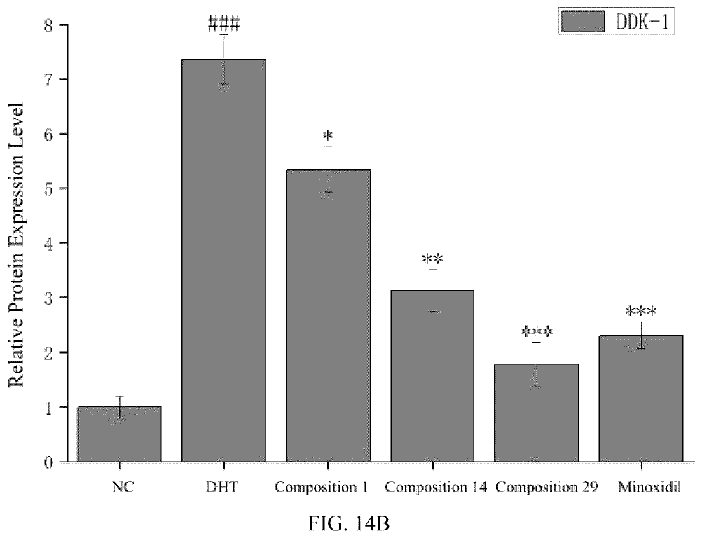

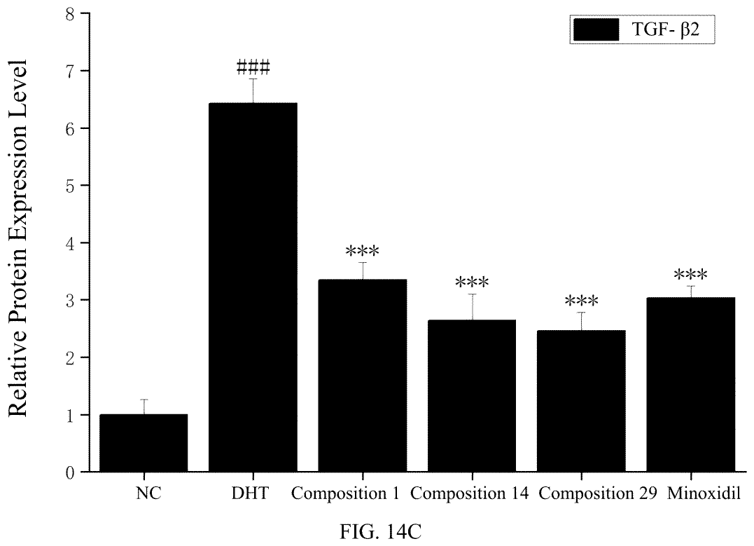

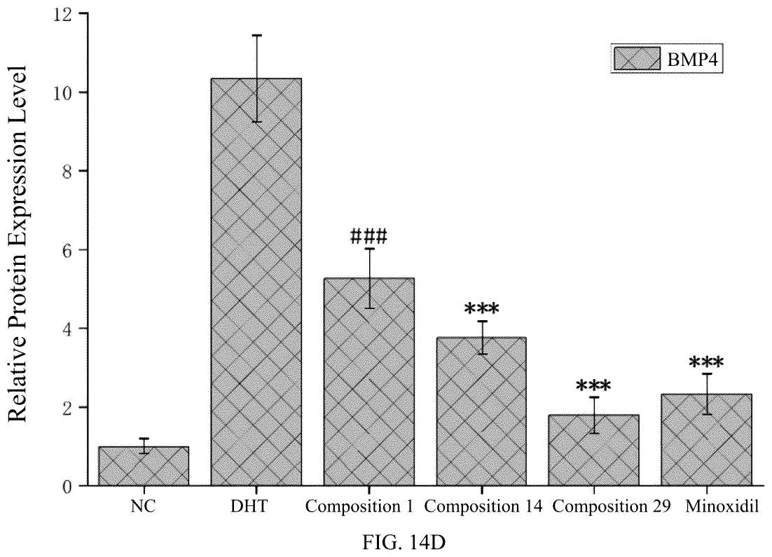

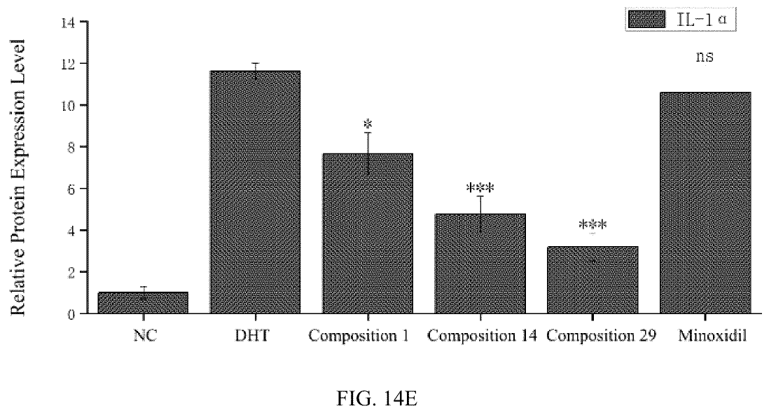

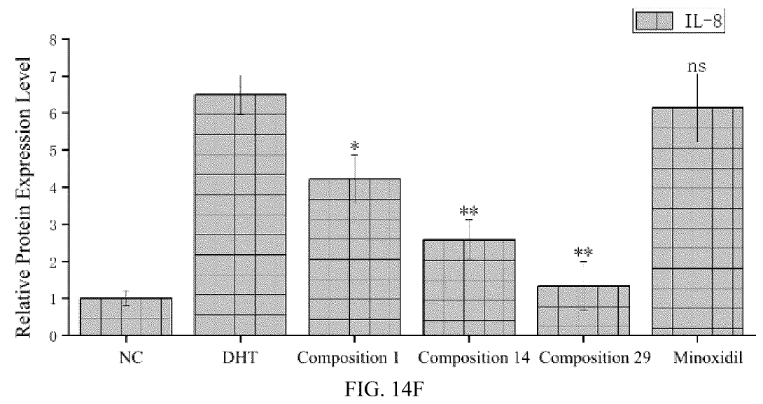

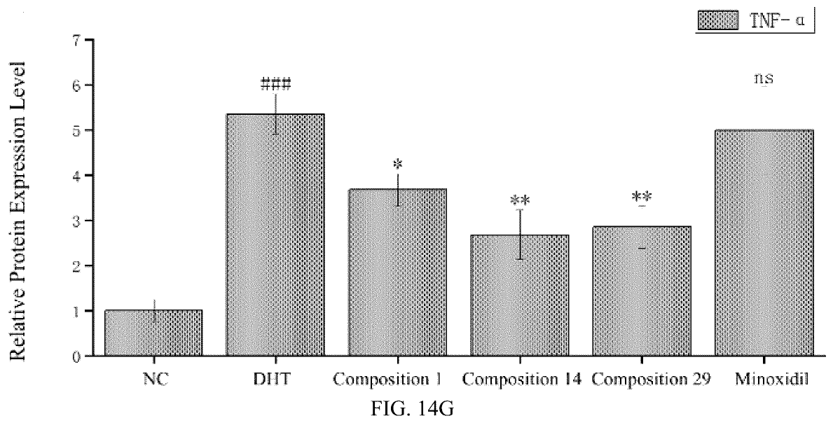

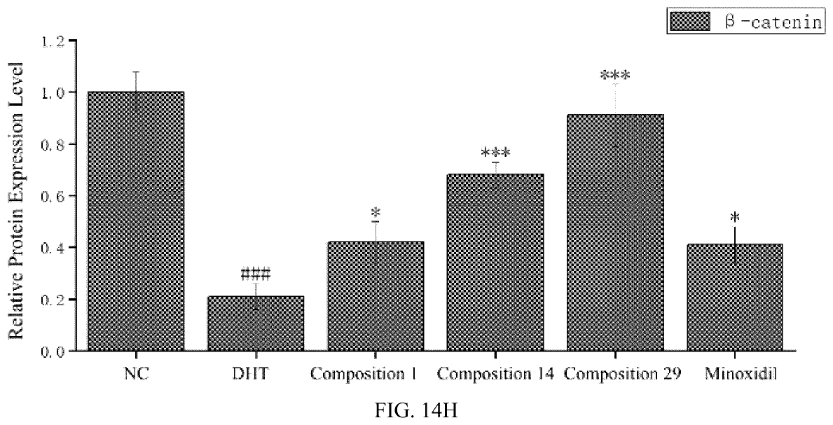

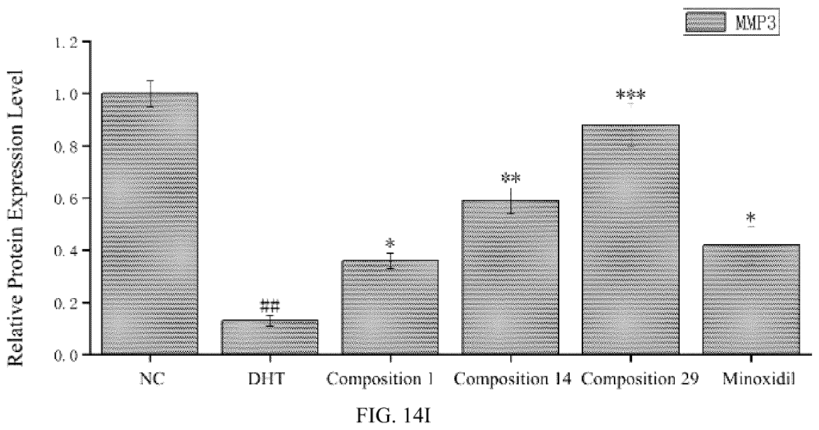

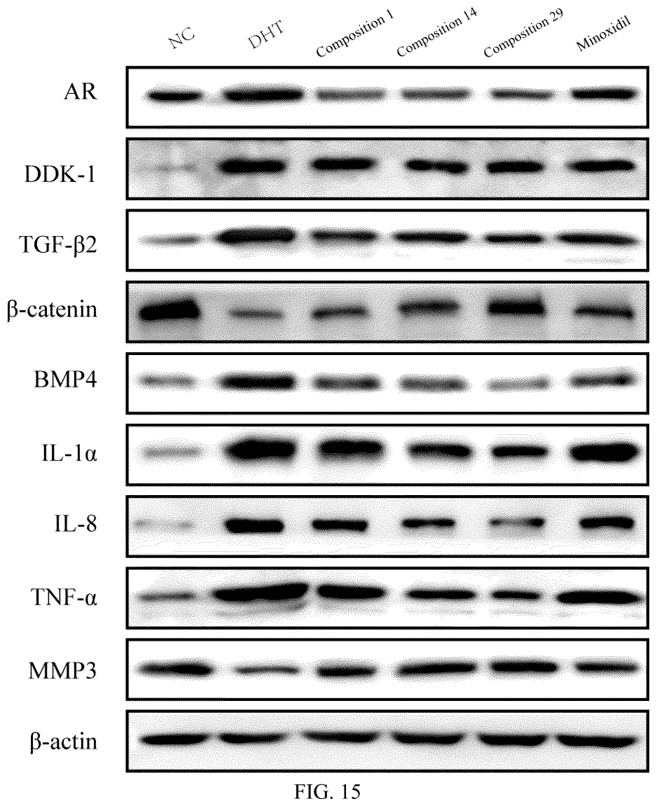

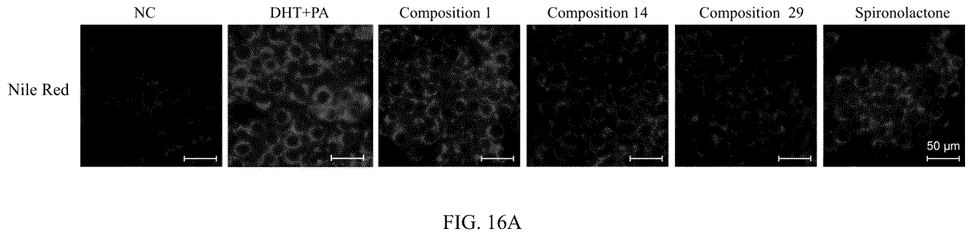

The influence of the compositions 1, 14, and 29 on the relative expression levels of the target protein AR and the related proteins DDK-1, TGF-β2, BMP4, IL-1α, IL-8, TNF-α, β-catenin, and MMP3 is shown in FIGS. 14A-14I. FIG. 14A is for AR, FIG. 14B is for DDK-1, FIG. 14C is for TGF-β2, FIG. 14D is for BMP4, FIG. 14E is for IL-1α, FIG. 14F is for IL-8, FIG. 14G is for TNF-α, FIG. 14H is for β-catenin, and FIG. 14I is for MMP3. Western blot results of influence of the compositions 1, 14, and 29 on the target protein AR and the related proteins IL-1α, IL-8, TNF-α, DKK-1, TGF-β2, BMP4, β-catenin, and MMP3 are shown in FIG. 15. In FIG. 15, NC represents normal HFDPCs, DHT represents DHT model cells, and compositions 1, 14, and 29 represent DHT model cells treated with the compositions 1, 14, and 29, respectively; P<0.05 is denoted as “*” or “#”; P<0.01 is denoted as “**” or “##”; and P<0.001 is denoted as “***” or “###”.

In summary, the compositions 1 to 37 of the present disclosure have a significant anti-hair loss effect.

### Test Example 6

Human immortalized sebaceous gland cells (SZ95) were selected as the target cells for oil control efficacy. SZ95 was stimulated with a combination of DHT (100 nM) and palmitic acid (PA, 50 μM) to establish an in vitro lipid accumulation cell model (DHT+PA model).

### 1. Testing of Oil Control Effects of Compositions for Cells

The influence of the compositions 1 to 37 of Example 2 or spironolactone (as a positive control) on the lipid deposition of DHT+PA model cells was investigated. The lipid deposition of SZ95 cells was detected by Nile red fluorescence assay. Nile Red was fully dissolved with anhydrous dimethyl sulfoxide (DMSO) to prepare a stock solution. The stock solution was then diluted with an HHBS buffer at a ratio of 1:1,000 to produce a 1×Nile Red working solution, which was vortexed for thorough mixing. The constructed DHT+PA model cells were treated with a composition or a positive control for 8 h, then centrifuged, and adjusted to a cell concentration of 1×10⁵ to 5×10⁵ cells/tube. Cells were resuspended with 500 μL of the Nile Red working solution and incubated at 37° C. in the dark for 10 min. The staining working solution was removed. Stained cells were washed with an HHBS buffer, and resuspended with 500 μL of a pre-heated HHBS buffer or medium at a cell density of 1×10⁵ to 5×10⁵ cells/tube. A fluorescence signal was detected by a fluorescence microscope, and Ex/Em=552 nm/636 nm.

According to test results, the DHT+PA model cells are successfully constructed. The DHT+PA treatment promotes the expression of lipids by SZ95 cells (Nile Red produces red fluorescence with lipids, and the stronger the red fluorescence, the higher the lipid content). However, in the presence of the compositions 1 to 37 or the positive control, a lipid content is significantly reduced. The semi-quantitative analysis results show that the compositions 14 and 29 exhibit a more significant effect for reducing the lipid deposition of SZ95 cells than the positive control spironolactone. It indicates that the compositions of the present disclosure have a significant oil control effect.

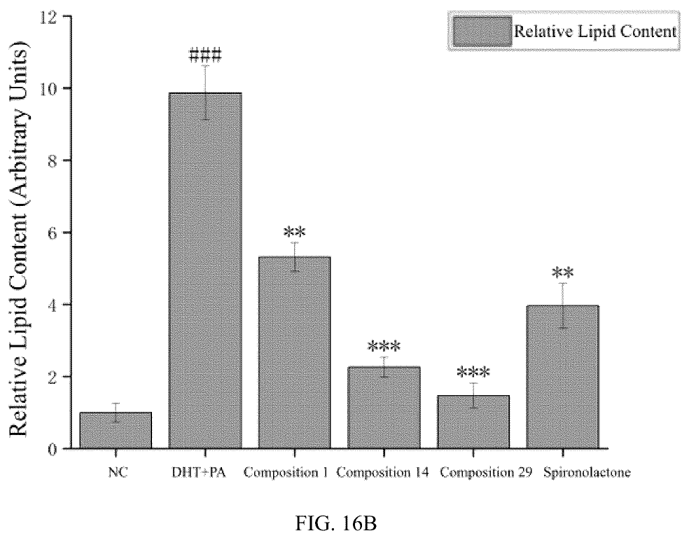

The influence of the compositions 1, 14, and 29 on lipids of SZ95 cells is shown in FIGS. 16A-16B. FIG. 16A shows the Nile red staining results; FIG. 16B shows the semi-quantitative analysis results of relative lipid contents; NC represents normal HFDPCs, DHT+PA represents DHT+PA model cells, and compositions 1, 14, and 29 represent DHT+PA model cells treated with the compositions 1, 14, and 29, respectively; P<0.05 is denoted as “*” or “#”; P<0.01 is denoted as “**” or “##”; and P<0.001 is denoted as “***” or “###”.

### 2. Influence of Compositions on Oil Control-Associated Proteins in Cells

The influence of the compositions 1 to 37 of Example 2 or spironolactone (as a positive control) on the expression of AR and proteins FASN, SREBP1c, CD36, and PPARα related to lipid synthesis and uptake or β-oxidation in DHT+PA model cells was investigated.

The compositions 1 to 37 with the same nucleic acid concentration (0.008%) each were added to the constructed DHT+PA model cells. Resulting mixtures each were incubated in a 37° C. and 5% CO₂ incubator for 8 h, and then centrifuged. Resulting cell pellets were collected, an RIPA lysis buffer was added, and lysis was allowed at 4° C. for 30 min. Centrifugation was conducted at 12,000 rpm for 10 min, and resulting supernatants were collected. The total cellular protein was extracted. The expression levels of the AR and the proteins related to lipid synthesis and uptake or β-oxidation were detected according to the protein detection method (Western blot) in Example 1.

According to test results, after the DHT+PA induction treatment, the expression levels of the AR and the lipid-associated proteins CD36, FASN, and SREBP1c in SZ95 cells significantly increase, while the expression level of the PPARα protein significantly decreases, indicating the successful modeling. After the DHT+PA model cells are treated with the compositions 1 to 37, the expression levels of AR, FASN, SREBP1c, and CD36 are significantly reduced compared with the model group, while the expression level of the PPARα protein significantly increases compared with the model group. After the treatment with the positive drug spironolactone, the expression levels of AR, FASN, SREBP1c, and CD36 significantly decrease, while the expression level of the PPARα protein significantly increases. The composition 29 exhibits a comparable effect to the positive control group, and even leads to a better effect than the positive control group in terms of PPARα protein expression. It indicates that the compositions of the present disclosure have a significant oil control effect.

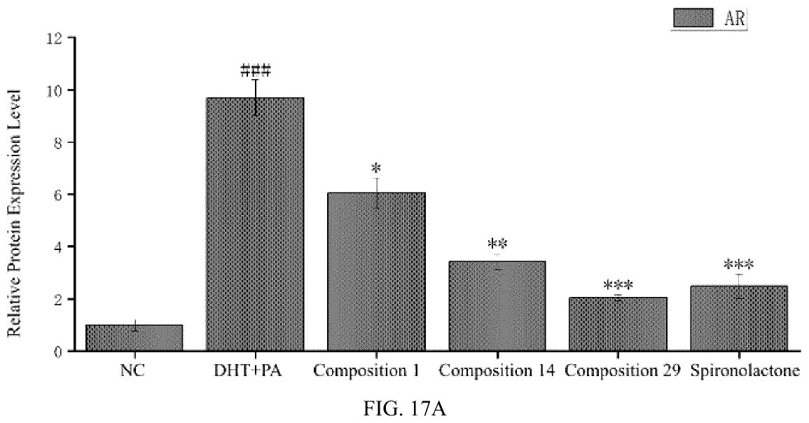

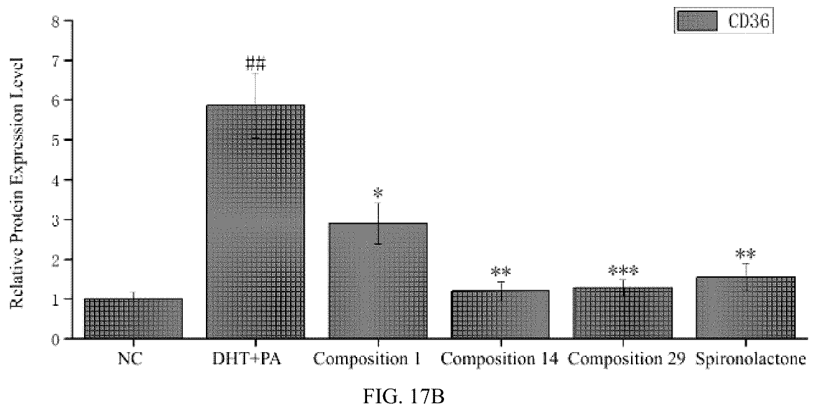

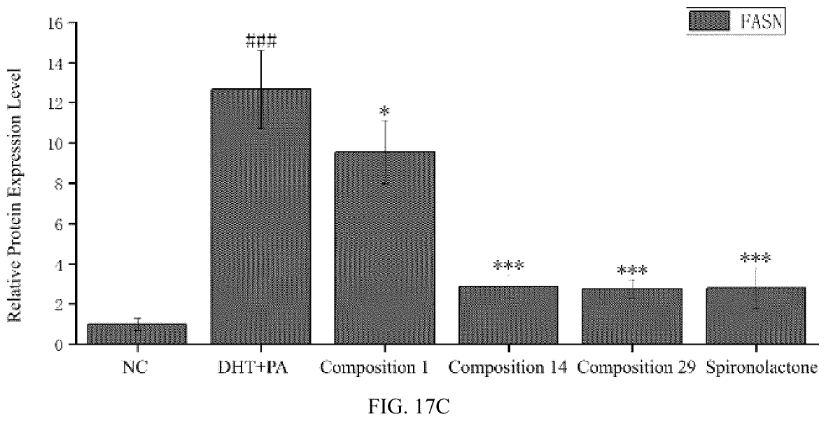

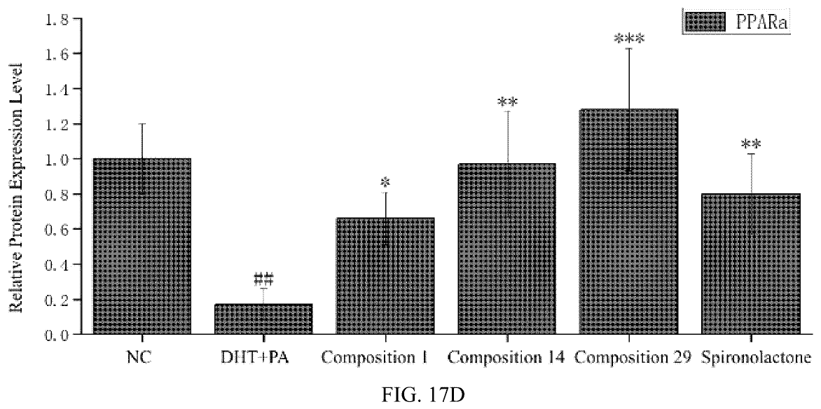

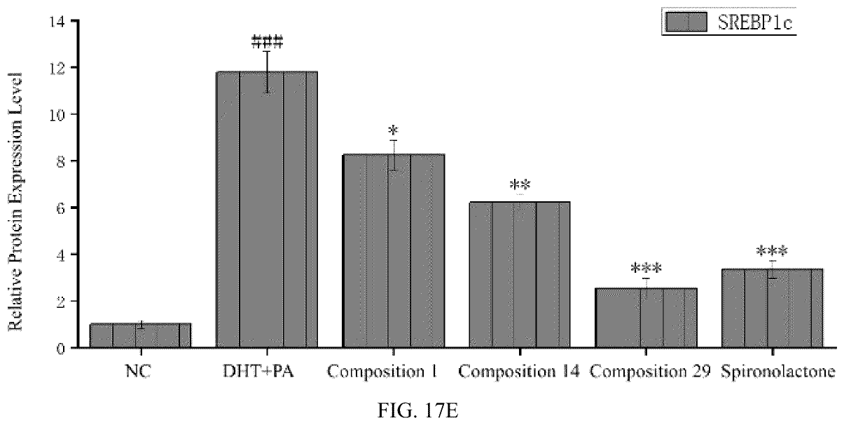

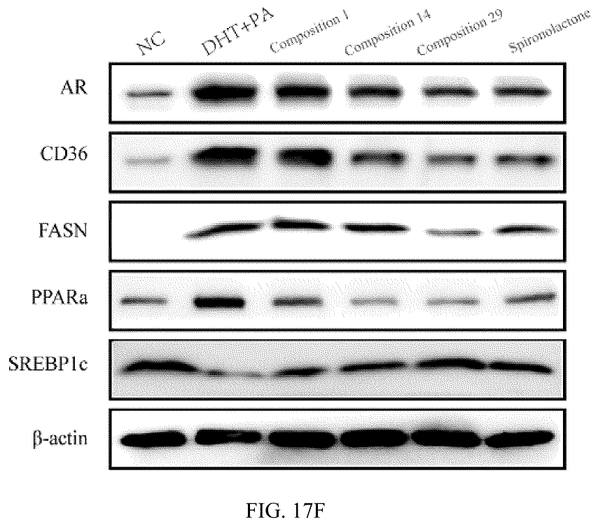

The influence of the compositions 1, 14, and 29 on the AR and lipid-associated proteins FASN, SREBP1c, CD36, and PPARα is shown in FIGS. 17A-17F. FIGS. 17A-17E show the relative expression levels of the proteins; FIG. 17F shows the western blot results; NC represents normal HFDPCs, DHT+PA represents DHT+PA model cells, and compositions 1, 14, and 29 represent DHT+PA model cells treated with the compositions 1, 14, and 29, respectively; P<0.05 is denoted as “*” or “#”; P<0.01 is denoted as “**” or “#”; and P<0.001 is denoted as “***” or “###”.

In summary, the compositions 1 to 37 of the present disclosure have a significant oil control effect.

### Test Example 7

An antibacterial activity was detected by a quantitative suspension test. The compositions 1 and 14 of Example 2 each were diluted 10 fold with PBS to produce sample solutions. The sample solutions and an ionic liquid (concentration: 50 ppm) each were mixed with a Propionibacterium acnes suspension, and an action was allowed for 1 min. The total number of Propionibacterium acnes cells was measured, and an inhibition rate was calculated in contrast to the control group to evaluate an antibacterial effect of a sample.

Propionibacterium acnes was cultured to the 4th to 8th generations, and colonies were collected and prepared with PBS into a microbial suspension with a concentration of 1× 10⁴ to 9×10⁴ CFU/mL. PBS was taken as a control. A composition sample to be tested was diluted with sterile standard hard water. 5.0 mL of a composition sample solution to be tested was pipetted and added to a sterilized test tube, then 0.1 mL of the microbial suspension was pipetted and added, and thorough mixing was allowed immediately for 1 min. 0.5 mL of a resulting mixed solution was taken and added to a test tube with 4.5 mL of sterilized PBS, and thorough mixing was allowed. The dilution was repeated twice. 1 mL of a mixed sample solution at each dilution ratio was pipetted and added to a sterilized petri dish, a medium was poured, and culturing was conducted. Colonies were counted. An inhibition rate was calculated based on a number of colonies in the blank control (a medium without a mixed sample solution).

The inhibition results of the compositions 1 and 14 and the ionic liquid against Propionibacterium acnes are shown in Table 6 below.

TABLE 6

| | Active ingredient | | Inhibition |
|---|---|---|---|
| Sample | concentration | | rate |
| Composition 1 | / | | 97.57% |
| Composition 14 | / | | 95.32% |
| Didecyldimethylammonium bromide | 50 | ppm | 99.97% |
| Didecyldimethylammonium chloride | 50 | ppm | 99.27% |

According to the results in Table 6, when diluted 10 fold, the compositions 1 and 14 exhibit a strong inhibitory effect on Propionibacterium acnes (90% or more has a significant antibacterial effect). It is also confirmed that didecyldimethylammonium bromide and didecyldimethylammonium chloride have a significant inhibitory effect on Propionibacterium acnes at a same concentration of 50 ppm. In combination with the significant anti-inflammatory effects for the inflammatory factors IL-1α, IL-8, and TNF-α proteins determined in the anti-hair loss test, these results further indicate that the compositions of the present disclosure have a potential anti-acne effect.

In summary, the compositions the present disclosure have significant anti-acne efficacy.

Finally, it should be noted that the above embodiments are provided merely to describe the technical solutions of the present disclosure, rather than to limit the protection scope of the present disclosure. Although the present disclosure is described in detail with reference to the preferred embodiments, those of ordinary skill in the art should understand that modifications or equivalent replacements may be made to the technical solutions of the present disclosure without departing from the spirit and scope of the technical solutions of the present disclosure.

1. A composition, comprising the following components in weight percentages: 0.00008% to 0.012% of a nucleic acid, 0.0002% to 5% of an ionic liquid, 0.0004% to 0.12% of a polymer, 0.011% to 0.33% of a lipid, 0.001% to 0.22% of a polypeptide, and a balance of a solvent, wherein the ionic liquid has a structural formula shown in a formula (I): 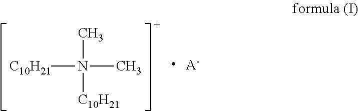
the formula (I),
wherein A: is selected from HCOO, CH₃COO⁻, CH₃CH₂COO⁻, CH₃CH₂CH₂COO⁻, Cl⁻, Br⁻, or NO₃⁻;
the polymer comprises at least one of polyethyleneimine, polyquaternium-7, polyarginine, or polylysine;
the solvent comprises water, an alcohol, or an ether;
the lipid comprises at least one of hydrogenated lecithin, soy lecithin,
phosphatidylethanolamine, dipalmitoylphosphatidylcholine, distearoylphosphatidylcholine, or cholesterol,
the polypeptide comprises at least one of acetyl tetrapeptide-3, acetyl dipeptide-1 cetyl ester, biotinoyl tripeptide-1, or oligopeptide-54;
the nucleic acid is a small interfering RNA (siRNA) targeting an androgen receptor (AR), and sequences of double strands of the nucleic acid are at least one group of the following or are produced through a chemical modification based on the at least one group of the following:
(1) the sequences shown in SEQ ID NO: 1 and SEQ ID NO: 2, respectively;
(2) the sequences shown in SEQ ID NO: 3 and SEQ ID NO: 4, respectively;
(3) the sequences shown in SEQ ID NO: 5 and SEQ ID NO: 6, respectively; and
the chemical modification comprises at least one of a modification of converting a nucleotide bond into a thiophosphate bond or a modification of combining the cholesterol.

2. A preparation method of the composition according to claim 1, comprising the following steps:
S1, dissolving the polymer, the nucleic acid, and the polypeptide with a first part of the solvent separately to produce a polymer solution, a nucleic acid solution, and a polypeptide solution, and refrigerating the polymer solution, the nucleic acid solution, and the polypeptide solution at 0° C. to 10° C. to obtain a refrigerated polymer solution, a refrigerated nucleic acid solution, and a refrigerated polypeptide solution;
S2, mixing the lipid, the ionic liquid, and a second part of the solvent, and allowing a complete dissolution at 40° C. to 60° C. to produce a mixture; adding a remaining part of the solvent at 40° C. to 60° C. to the mixture, and stirring for 5 min to 10 min; conducting a high-speed shearing at 3,000 r/min to 5,000 r/min for 1 min to 3 min, and conducting a high-pressure homogenization 2 times to 6 times at 200 bar to 600 bar to produce a homogeneous mixture; and refrigerating the homogeneous mixture at 0° C. to 10° C. to obtain a refrigerated homogeneous mixture; and
S3, adding the refrigerated polymer solution obtained in the step S1 to the refrigerated homogeneous mixture obtained in the step S2, and thoroughly mixing; adding the refrigerated polypeptide solution obtained in the step S1, and thoroughly mixing; and adding the refrigerated nucleic acid solution obtained in the step S1, and incubating for 1 h to 4 h to produce the composition.

3. A use of the composition according to claim 1 in a preparation of a product with an anti-hair loss effect and an oil control effect.

4. A product with an anti-hair loss effect and an oil control effect, comprising the composition according to claim 1.
